SEQUENCE LISTING (64 sequences)

SEQ ID NO: 1 - protein, 13 aa, Artificial Sequence
  Synthetic polypeptide
APGDRGEPGPPGP

SEQ ID NO: 2 - protein, 13 aa, Artificial Sequence
  Synthetic polypeptide
APGDRGEPGPPGP

SEQ ID NO: 3 - protein, 14 aa, Artificial Sequence
  Synthetic polypeptide
APGDRGEPGPPGPA

SEQ ID NO: 4 - protein, 15 aa, Artificial Sequence
  Synthetic polypeptide
DAGPVGPPGPPGPPG

SEQ ID NO: 5 - protein, 16 aa, Artificial Sequence
  Synthetic polypeptide
GPPGPPGPPGPPGPPS

SEQ ID NO: 6 - protein, 21 aa, Artificial Sequence
  Synthetic polypeptide
NGDDGEAGKPGRPGERGPPGP

SEQ ID NO: 7 - protein, 21 aa, Artificial Sequence
  Synthetic polypeptide
NGDDGEAGKPGRPGERGPPGP

SEQ ID NO: 8 - protein, 22 aa, Artificial Sequence
  Synthetic polypeptide
NGDDGEAGKPGRPGERGPPGPQ

SEQ ID NO: 9 - protein, 24 aa, Artificial Sequence
  Synthetic polypeptide
GKNGDDGEAGKPGRPGERGPPGPQ

SEQ ID NO: 10 - protein, 24 aa, Artificial Sequence
  Synthetic polypeptide
GKNGDDGEAGKPGRPGERGPPGPQ

SEQ ID NO: 11 - protein, 27 aa, Artificial Sequence
  Synthetic polypeptide
GPPGKNGDDGEAGKPGRPGERGPPGPQ

SEQ ID NO: 12 - protein, 18 aa, Artificial Sequence
  Synthetic polypeptide
PPGEAGKPGEQGVPGDLG

SEQ ID NO: 13 - protein, 22 aa, Artificial Sequence
  Synthetic polypeptide
PPGEAGKPGEQGVPGDLGAPGP

SEQ ID NO: 14 - protein, 25 aa, Artificial Sequence
  Synthetic polypeptide
ADGQPGAKGEPGDAGAKGDAGPPGP

SEQ ID NO: 15 - protein, 25 aa, Artificial Sequence
  Synthetic polypeptide
ADGQPGAKGEPGDAGAKGDAGPPGP

SEQ ID NO: 16 - protein, 26 aa, Artificial Sequence
  Synthetic polypeptide
ADGQPGAKGEPGDAGAKGDAGPPGPA

SEQ ID NO: 17 - protein, 26 aa, Artificial Sequence
  Synthetic polypeptide
ADGQPGAKGEPGDAGAKGDAGPPGPA

SEQ ID NO: 18 - protein, 30 aa, Artificial Sequence
  Synthetic polypeptide
GPPGADGQPGAKGEPGDAGAKGDAGPPGPA

SEQ ID NO: 19 - protein, 22 aa, Artificial Sequence
  Synthetic polypeptide
EGSPGRDGSPGAKGDRGETGPA

SEQ ID NO: 20 - protein, 23 aa, Artificial Sequence
  Synthetic polypeptide
AEGSPGRDGSPGAKGDRGETGPA

SEQ ID NO: 21 - protein, 32 aa, Artificial Sequence
  Synthetic polypeptide
ESGREGAPGAEGSPGRDGSPGAKGDRGETGPA

SEQ ID NO: 22 - protein, 14 aa, Artificial Sequence
  Synthetic polypeptide
SPGPDGKTGPPGPA

SEQ ID NO: 23 - protein, 23 aa, Artificial Sequence
  Synthetic polypeptide
DGKTGPPGPAGQDGRPGPPGPPG

SEQ ID NO: 24 - protein, 22 aa, Artificial Sequence
  Synthetic polypeptide
GRPGEVGPPGPPGPAGEKGSPG

SEQ ID NO: 25 - protein, 20 aa, Artificial Sequence
  Synthetic polypeptide
DGPPGRDGQPGHKGERGYPG

SEQ ID NO: 26 - protein, 21 aa, Artificial Sequence
  Synthetic polypeptide
NDGPPGRDGQPGHKGERGYPG

SEQ ID NO: 27 - protein, 20 aa, Artificial Sequence
  Synthetic polypeptide
SNGNPGPPGPPGPSGKDGPK

SEQ ID NO: 28 - protein, 19 aa, Artificial Sequence
  Synthetic polypeptide
NDGAPGKNGERGGPGGPGP

SEQ ID NO: 29 - protein, 20 aa, Artificial Sequence
  Synthetic polypeptide
DGESGRPGRPGERGLPGPPG

SEQ ID NO: 30 - protein, 24 aa, Artificial Sequence
  Synthetic polypeptide
DAGAPGAPGGKGDAGAPGERGPPG

SEQ ID NO: 31 - protein, 28 aa, Artificial Sequence
  Synthetic polypeptide
GAPGQNGEPGGKGERGAPGEKGEGGPPG

SEQ ID NO: 32 - protein, 30 aa, Artificial Sequence
  Synthetic polypeptide
KNGETGPQGPPGPTGPGGDKGDTGPPGPQG

SEQ ID NO: 33 - protein, 15 aa, Artificial Sequence
  Synthetic polypeptide
PGXQGNPGAXGLPGP

SEQ ID NO: 34 - protein, 12 aa, Artificial Sequence
  Synthetic polypeptide
GLPGLPGPKGFA

SEQ ID NO: 35 - protein, 13 aa, Artificial Sequence
  Synthetic polypeptide
GEPGPPGPPGNLG

SEQ ID NO: 36 - protein, 13 aa, Artificial Sequence
  Synthetic polypeptide
GLPGPPGPKGPRG

SEQ ID NO: 37 - protein, 13 aa, Artificial Sequence
  Synthetic polypeptide
GPPGPPGPLGPLG

SEQ ID NO: 38 - protein, 13 aa, Artificial Sequence
  Synthetic polypeptide
PGLXGMKGDPGLP

SEQ ID NO: 39 - protein, 18 aa, Artificial Sequence
  Synthetic polypeptide
GIKGEKGXPGXPGLPGLP

SEQ ID NO: 40 - protein, 13 aa, Artificial Sequence
  Synthetic polypeptide
GLPGPPGPPGPPS

SEQ ID NO: 41 - protein, 19 aa, Artificial Sequence
  Synthetic polypeptide
KGPXGKPGLAGMPGAXGPP

SEQ ID NO: 42 - protein, 18 aa, Artificial Sequence
  Synthetic polypeptide
PGLPGQVGETGKPGAPGR

SEQ ID NO: 43 - protein, 18 aa, Artificial Sequence
  Synthetic polypeptide
KRPDSGATGLPGRPGPPG

SEQ ID NO: 44 - protein, 16 aa, Artificial Sequence
  Synthetic polypeptide
GPPGPPGLPGPQGPKG

SEQ ID NO: 45 - protein, 19 aa, Artificial Sequence
  Synthetic polypeptide
DGPPGPPGERGPQGPQGPV

SEQ ID NO: 46 - protein, 14 aa, Artificial Sequence
  Synthetic polypeptide
LPGPPGPPGSFLSN

SEQ ID NO: 47 - protein, 13 aa, Artificial Sequence
  Synthetic polypeptide
GPPGPPGPPGPPS

SEQ ID NO: 48 - protein, 9 aa, Artificial Sequence
  Synthetic polypeptide
VLNLGPITR

SEQ ID NO: 49 - protein, 10 aa, Artificial Sequence
  Synthetic polypeptide
SGSVIDQSRV

SEQ ID NO: 50 - protein, 11 aa, Artificial Sequence
  Synthetic polypeptide
DQSRVLNLGPI

SEQ ID NO: 51 - protein, 11 aa, Artificial Sequence
  Synthetic polypeptide
SRVLNLGPITR

SEQ ID NO: 52 - protein, 12 aa, Artificial Sequence
  Synthetic polypeptide
IDQSRVLNLGPI

SEQ ID NO: 53 - protein, 13 aa, Artificial Sequence
  Synthetic polypeptide
VIDQSRVLNLGPI

SEQ ID NO: 54 - protein, 13 aa, Artificial Sequence
  Synthetic polypeptide
DQSRVLNLGPITR

SEQ ID NO: 55 - protein, 14 aa, Artificial Sequence
  Synthetic polypeptide
SVIDQSRVLNLGPI

SEQ ID NO: 56 - protein, 15 aa, Artificial Sequence
  Synthetic polypeptide
GSVIDQSRVLNLGPI

SEQ ID NO: 57 - protein, 14 aa, Artificial Sequence
  Synthetic polypeptide
IDQSRVLNLGPITR

SEQ ID NO: 58 - protein, 16 aa, Artificial Sequence
  Synthetic polypeptide
SGSVIDQSRVLNLGPI

SEQ ID NO: 59 - protein, 15 aa, Artificial Sequence
  Synthetic polypeptide
VIDQSRVLNLGPITR

SEQ ID NO: 60 - protein, 17 aa, Artificial Sequence
  Synthetic polypeptide
SGSVIDQSRVLNLGPIT

SEQ ID NO: 61 - protein, 16 aa, Artificial Sequence
  Synthetic polypeptide
SVIDQSRVLNLGPITR

SEQ ID NO: 62 - protein, 18 aa, Artificial Sequence
  Synthetic polypeptide
SGSVIDQSRVLNLGPITR

SEQ ID NO: 63 - protein, 19 aa, Artificial Sequence
  Synthetic polypeptide
SGSVIDQSRVLNLGPITRK

SEQ ID NO: 64 - protein, 640 aa, Artificial Sequence
  Synthetic polypeptide
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

(19) United States
(12) Patent Application Publication
(10) Pub. No.: US 2012/0283123 A1
(43) Pub. Date: Nov. 8, 2012

(54) Biomarkers for the Diagnosis of Kidney Graft Rejection

(76) Inventors: Minnie M. Sarwal, Portola Valley, CA (US); Bruce Xuefeng Ling, Palo alto, CA (US); Tara Sigdel, Palo Alto, CA (US); James Schilling, San Mateo, CA (US)

(21) Appl. No.: 13/508,013

(22) PCT Filed: Nov. 24, 2010
(86) PCT No.: PCT/US10/57994

§ 371 Date: Jul. 19, 2012

Related U.S. Application Data
(60) Provisional application No. 61/283,093, filed on Nov. 25, 2009.

(51) Int. Cl. G01N 27/72; C40B 30/04; C40B 40/06; C12Q 1/68

(52) U.S. Cl. 506/9; 435/6.12; 436/86; 506/16

(57) ABSTRACT

Methods are provided for determining a transplant category of a subject having a kidney graft. In practicing one aspect of the subject methods, the peptide signature of a non-invasive sample derived from the transplant subject (e.g., a urine sample) is used to determine the subject's transplant category (e.g., acute allograft rejection (AR), stable allograft (STA), BK virus nephropathy (BK), and the like). In other embodiments, a gene expression signature from a biopsy sample from the subject (e.g., mRNA level) is used to determine the subject's transplant category. In certain embodiments both a peptide signature and a gene expression signature are used. Also provided are compositions, systems, kits and computer program products that find use in practicing the subject methods.

### GOVERNMENT RIGHTS

This invention was made with Government support under contract AI-075256 awarded by the National Institutes of Health. The Government has certain rights in this invention.

### BACKGROUND

Transplantation of a graft organ or tissue from a donor to a host patient is a feature of certain medical procedures and treatment protocols. Despite efforts to avoid graft rejection through host-donor tissue type matching, in transplantation procedures where a donor organ is introduced into a host, immunosuppressive therapy is generally required to the maintain viability of the donor organ in the host. However, despite the wide use of immunosuppressive therapy, organ transplant rejection can occur.

Acute graft rejection (AR) of allograft tissue is a complex immune response that involves T-cell recognition of alloantigen in the allograft, co-stimulatory signals, elaboration of effectors molecules by activated T cells, and an inflammatory response within the graft. Activation and recruitment of circulating leukocytes to the allograft is a central feature of this process.

Early detection of AR is one of the major clinical concerns in the care of transplant recipients, including kidney transplant recipients. Detection of AR before the onset of renal dysfunction allows successful treatment of this condition with aggressive immunosuppression. It is equally important to reduce immunosuppression in patients who do not have AR to minimize drug toxicity.

Accordingly, techniques for monitoring for an AR response in a transplant recipient, including predicting, diagnosing and characterizing AR, are of interest in the field. The present invention meets these and other needs.

### SUMMARY OF THE INVENTION

Methods are provided for determining a transplant category of a subject having a kidney graft. In practicing one aspect of the subject methods, the peptide signature of a non-invasive sample derived from the transplant subject (e.g., a urine sample) is used to determine the subject's transplant category (e.g., acute allograft rejection (AR), stable allograft (STA), BK virus nephropathy (BK), and the like). In other embodiments, a gene expression signature from a biopsy sample from the subject (e.g., mRNA level) is used to determine the subject's transplant category. In certain embodiments both a peptide signature and a gene expression signature are used. Also provided are compositions, systems, kits and computer program products that find use in practicing the subject methods. The methods and compositions find use in a variety of applications.

### DEFINITIONS

For convenience, certain terms employed in the specification, examples, and appended claims are collected here.

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or CR” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerosis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “transplant rejection” encompasses both acute and chronic transplant rejection.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

As used herein, the term “gene” or “recombinant gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, procaryotic or eukaryotic mRNA, genomic DNA sequences from viral, procaryotic or eukaryotic DNA, and even synthetic DNA sequences. A transcription termination sequence may be located 3′ to the coding sequence.

The terms “reference” and “control” are used interchangebly to refer to a known value or set of known values against which an observed value may be compared. As used herein, known means that the value represents an understood parameter, e.g., a level of expression of a marker gene in a graft survival or loss phenotype.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides.

The terms “protein”, “polypeptide”, “peptide” and the like refer to a polymer of amino acids (an amino acid sequence) and does not refer to a specific length of the molecule. This term also refers to or includes any modifications of the polypeptide (e.g., post-translational), such as glycosylations, acetylations, phosphorylations and the like. Included within the definition are, for example, polypeptides containing one or more analogs of an amino acid, polypeptides with substituted linkages, as well as other modifications known in the art, both naturally occurring and non-naturally occurring.

The term “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

The terms “profile” and “signature” and “result” and “data”, and the like, when used to describe peptide level or gene expression level data are used interchangeably (e.g., peptide signature/profile/result/data, gene expression signature/profile/result/data, etc.).

Certain abbreviations employed in this application include the following:

AR: Acute Rejection;

AUC: Area under the curve;

AZA: Aza thioprine;

BK: BK-virus nephropathy;

BKV: BK-strain of Polyoma virus;

CMV: Cytomegalovirus;

FDR: false discovery rate;

HC: Healthy control (e.g., a non-transplant recipient);

HPLC: high performance liquid chromatography;

LC: Liquid chromatography (e.g., HPLC);

LC-MS: Liquid chromatography and mass spectroscopy;

LC-MALDI: Liquid chromatography and matrix-assisted laser desorption ionization;

LDA: linear discriminant analysis

MALDI: matrix-assisted laser desorption ionization;

MS: mass spectroscopy

NS: non-specific proteinuria with native renal diseases; nephrotic syndrome;

NSC: nearest shrunken centroid classifiers;

PBL: Peripheral Blood Leukocytes;

PAM: Prediction Analysis of Microarrays;

Q-PCR: quantitative real time polymerase chain reaction;

ROC: Receiver Operating Characteristic;

SAM: Significance Analysis of Microarrays;

STA: stable allograft;

WBC: White blood cell.

### BRIEF DESCRIPTION OF THE FIGURES

FIG. 1. Peptidomics approach for biomarker discovery. (A) Schematics for peptidomic analysis of naturally occurring urinary peptides. A flowchart for urinary peptide extraction and processing by LC MALDI method is shown. (B) Study design for the urine peptide biomarker discovery.

FIG. 2. Statistical analyses of the 40 peptide biomarker panel. (A) The discriminant of the peptide biomarker panel for the training (upper) and testing data (lower) probabilities for all transplant samples were calculated from the linear discriminant analysis (LDA). The maximum estimated probability for each of the wrongly classified samples is marked with a circle. 2 samples of the 46 samples in the training-set and 4 of the 24 samples in the test-set were misclassified, giving a correct classification rate of 96% in the training-set and 83% in the test-set.

(B) Left panel: Modified 2×2 contingency tables were used to calculate the percentage of classification that agreed with clinical diagnosis for the biomarker panel. P-values were calculated with Fisher's exact test. Right panel: A prediction of AR from non-AR phenotype (a so-called “two-class” prediction) has been utilized to assess the performance of the biomarker panel in the classification of unknown samples. STA and BK were combined into one group as “NON-AR”. Fisher exact test was to compute the P value for the blind test.

(C) Unsupervised clustering based upon the 53 peptide panel was used to construct a heatmap, where the colors indicate the intensity of peptide concentration by LC-MALDI; red indicates high peptide abundance and green indicates low peptide abundance in the comparative analysis. It can be seen that by unsupervised analysis, the AR samples, save one, all co-cluster together, and all of the non-AR samples cluster together. Modified 2×2 contingency tables were used to calculate the percentage of unsupervised clustering that agreed with clinical diagnosis for the biomarker panel. P-values were calculated with Fisher's exact test.

FIG. 3. (A) Discovery of 40 peptide biomarker panel and their performance on the training set (top panel) and the test set (bottom panel) using ROC analysis. (B) MRM analyses of the two UMOD peptide biomarkers (top panels). For the UMOD1 peptide (1680.98 Da), the prominent precursor ion is the triply charged 563.7 ion and the most prominent product ion is the doubly charged y13 735.5 ion, and for the UMOD2 peptide (1912.07 Da), the prominent precursor ion is the triply charged 638.4 ion and the most prominent product ion is the doubly charged y14 791.9 ion. The distribution of MRM signals were analyzed by box-whisker graphs according to the sample categories. The boxes are bound by 75^{th} and 25^{th} percentiles of the data and the whiskers extend to the minimum and maximum values. ROC analysis (bottom panel) of the classification performance of the two UMOD peptide biomarkers. AUC: area under curve. When ROC analysis was performed to test the diagnostic accuracy of the two UMOD peptide biomarkers for AR, the AUCs were computed as 0.83 for the UMOD 1680.98 peptide and 0.74 for the UMOD 1912.07 peptide.

FIG. 4: (A) The distribution of COL1A2, COL3A1, MMP7, SERPING1, TIMP1, and UMOD genes' RT-PCR measurements in kidney biopsy were analyzed by box-whisker graphs. (B) ROC analysis to evaluate the performance of the 7 member RNA biomarker panel classifying AR from STA.

FIG. 5. A proposed mechanism of fibrosis caused by AR as indicated by the observations of increased collagen gene transcription in the rejection biopsy and reduced collagen peptides in the urine during graft rejection.

FIG. 6. Six fold cross-validation analysis led to the discovery of a set of 630 features with lowest possible classification error. In internal cross-validation, decreasing the centroid threshold (lower x-axis) resulted in an increase in the number of markers (inserted upper x-axis) that were used for classification and calculation of the classification error (y-axis).

FIG. 7. Analyses of the discriminant class probabilities for the 630 feature biomarker panel. Discriminant class probabilities and Gaussian linear discriminant analysis were calculated for each sample (top panel: training samples; bottom panel: testing samples). With the maximum estimated probability marked with a circle, one of the AR test samples are predicted correctly with low confidence, and one STA test sample are wrongly classified as BK. All the nephrotic syndrome and healthy control samples were included in the training set.

FIG. 8. Goodness of separation analysis for each tested nearest shrunken centroid (NSC) classifiers. In this study, the goodness of separation is defined by computing the difference of the discriminative scores (estimated probability [16]): if predicted correctly, Δ probability is the difference of the highest and next highest probability; if predicted incorrectly, probability is the difference of the true class's probability and the highest probability, which will be negative. For each panel, whisker plots for AR, STA, and BK were generated. The analysis of the goodness of separation revealed 53 to be the smallest panel size, where in both training and testing cases the “box” values of goodness of separation of all AR, STA and BK categories remain positive. 40 of the 53 peptides have been identified.

FIG. 9. Peptidomic analysis of UMOD and various collagens. A log of ratio of peptide level in AR to stable/healthy urine. Between AR and HC, the logarithmic ratios of the medians of the peptide protein precursors were calculated, and the distribution was plotted as box-whisker graphs. All peptide biomarkers coming from the same precursor UMOD, COL1A1, COL1A2, COL2A1, COL3A1, COL4A1, COL4A2, COL4A3, COL4A4, COL4A5, COL4A6, COL7A1, COL9A1, COL11A1, COL17A1 and COL18A1 were of lower abundance in AR urine. However, the expression of COL1A2, COL3A1, COL4A1, MMP7, SERPING1 and TIMP1 (FIG. 4A and FIG. 9) are up regulated in AR. All of these observations suggest that dysfunction of proteolytic pathways in AR and up regulation of collagens lead to accumulation of collagens in allograft, which ultimately results fibrosis and allograft dysfunction and rejection.

Table 2. Demographical summary of patient groups for urine peptide biomarker discovery.

### DESCRIPTION OF THE SPECIFIC EMBODIMENTS

Aspects of the subject invention provide methods for determining a clinical transplant category of a subject who has received a kidney transplant. In certain embodiments, the methods include obtaining a sample non-invasively from the subject (e.g., urine) and determining the level of one or more peptides therein to obtain a peptide signature of the sample. The peptide signature can then be used to determine the clinical transplant category of the subject, e.g., by comparing to one or more peptide signatures from subjects having a known transplant category (e.g., acute rejection, stable, non-transplant, etc.). Such known peptide signatures can also be called controls. In certain other embodiments, the level of expression of at least one gene in a biopsy sample from the subject is determined to obtain gene expression signature of the biopsy sample. The gene expression result can measure any gene product or activity of the gene of interest, e.g., mRNA level, protein level, enzymatic activity, etc. The gene expression signature can then be used to determine the clinical transplant category of the subject, e.g., by comparing to one or more gene expression signatures from subjects having a known transplant category (e.g., acute rejection, stable, non-transplant, etc.). In certain embodiments, both a peptide signature from a non-invasive sample and a gene expression signature from a biopsy sample of the subject are used to determine the transplant category. Also provided are compositions, systems, kits and computer program products that find use in practicing the subject methods.

Aspects of the subject invention include methods of determining the clinical transplant category of a subject who has received a kidney transplant. Clinical transplant categories include: acute rejection (AR) response; stable allograft (STA); BK virus nephropathy (BK), and the like.

In certain embodiments the method includes: (a) evaluating the amount of one or more peptides in a non-invasive sample from a transplant subject to obtain a peptide signature; and (b) employing the peptide signature to determine the transplant category of the subject. In certain embodiments, the peptide signature comprises peptide amount data for one or more peptides in Tables 1A and/or 1B.

In certain embodiments, the method includes: (a) evaluating the gene expression level of one or more genes in a biopsy sample from a transplant subject to obtain a gene expression signature; and (b) employing the gene expression signature to determine the transplant category of the subject. In certain embodiments, the gene expression signature comprises data for one or more of the following genes: COL1A2, COL3A1, MMP7, SERPING1, TIMP1 and UMOD. In certain embodiments, the gene expression signature includes data for all of these genes.

In certain embodiments, both a peptide signature and a gene expression signature are employed to determine the transplant category of the subject. In certain embodiments, the methods can be employed to monitor a subject over time for transplant category and/or be used to determine a treatment regimen for the subject (e.g., whether or not modulation of immunosuppressive therapy for the subject is indicated).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the subject invention provide methods for determining a clinical transplant category of a subject who has received a kidney transplant, as well as reagents, systems, kits and computer program products for use in practicing the subject methods. In further describing the invention, the subject methods are described first, followed by a review of the reagents, systems, kits and computer program products for use in practicing the subject methods.

### Methods for Determining a Clinical Transplant Category

Aspects of the subject invention include methods for determining a clinical transplant category of a subject who has received a kidney transplant.

As is known in the transplantation field, a graft organ, tissue or cell(s) may be allogeneic or xenogeneic, such that the grafts may be allografts or xenografts.

In certain embodiments, the method can be considered a method of monitoring a subject to determine a clinical transplant category, e.g., at one or more time points after kidney transplantation. Clinical transplant categories that can be determine using the methods of the subject invention include, but are not limited to: acute allograft rejection (AR), stable allograft (STA), and BK-virus nephropathy (BK). In certain embodiments, the subject methods distinguish one or more of the clinical transplant categories from non-transplant kidney categories, including subjects with non-specific proteinuria with native renal diseases (nephrotic syndrome, or NS), subjects healthy kidney function (HC), etc.

In certain embodiments, a subject is monitored non-invasively to determine clinical transplant category. By “non-invasively” is meant that the sample from the subject to determine a clinical transplant category is obtained via non-surgical methods, i.e., the sample is not obtained by harvesting tissue, blood, serum, etc., using a needle, scalpel, or other surgical tool employed for invasive tissue/sample harvesting. In certain embodiments, the non-invasively obtained sample is selected from urine, saliva, and tears, where in certain embodiments the non-invasive sample is a urine sample.

In practicing the subject methods, the non-invasively procured sample is assayed to obtain a peptide signature of the sample, or peptide profile, in which the amount of one or more specific peptides in the sample is determined, where the determined amount may be relative and/or quantitative in nature. In certain embodiments, the peptide signature includes measurements for the amount of one or more peptides shown in Tables 1A and 1B. The high resolution mass spectrometric analysis uncovered 53 mass spectrometric peaks discriminating different allograft dysfunction classes. Subsequent deconvoluting and deisotoping analysis found 40 unique peptides from these 53 peaks, upon which a mathematic model was developed as a classifier to discriminate different allograft dysfunctions (AR, STA and BK). Urine naturally occurring peptide catalog analysis found that different overlapping peptides (total of 63 peptides, Table 1A and 1B) cluster with differential disease predictive power. The term peptide profile is used broadly to include a profile of one or more different peptides in the sample, where the peptides are derived from expression products of one or more genes. As such, in certain embodiments, the level of only one peptide shown in Tables 1A or 1B is evaluated. In yet other embodiments, the level of two or more peptides from Tables 1A or 1B is evaluated, e.g., 3, 4, 5, 10, 20, 30, 40, 50 or all 63 peptides listed in Tables 1A and 1B. In certain embodiments, the expression level of one or more additional peptides other than those listed in Tables 1A and 1B is also evaluated.

TABLE 1A
Collagen-derived Peptides

| | SEQ | | | |
|---|---|---|---|---|
| | ID | Precursor | | |
| S.No | NO: | Gene | M/Z | Peptide Sequence |
| 1 | 1 | COL1A1 | 1235.56 | APGDRGEPGPPGP |
| 2 | 2 | COL1A1 | 1251.55 | APGDRGEPGPPGP |
| 3 | 3 | COL1A1 | 1322.57 | APGDRGEPGPPGPA |
| 4 | 4 | COL1A1 | 1316.59 | DAGPVGPPGPPGPPG |
| 5 | 5 | COL1A1 | 1409.66 | GPPGPPGPPGPPGPPS |
| 6 | 6 | COL1A1 | 2048.92 | NGDDGEAGKPGRPGERGPPGP |
| 7 | 7 | COL1A1 | 2064.91 | NGDDGEAGKPGRPGERGPPGP |
| 8 | 8 | COL1A1 | 2192.97 | NGDDGEAGKPGRPGERGPPGPQ |
| 9 | 9 | COL1A1 | 2362.12 | GKNGDDGEAGKPGRPGERGPPGPQ |
| 10 | 10 | COL1A1 | 2378.10 | GKNGDDGEAGKPGRPGERGPPGPQ |
| 11 | 11 | COL1A1 | 2645.24 | GPPGKNGDDGEAGKPGRPGERGPPGPQ |
| 12 | 12 | COL1A1 | 1709.79 | PPGEAGKPGEQGVPGDLG |
| 13 | 13 | COL1A1 | 2031.95 | PPGEAGKPGEQGVPGDLGAPGP |
| 14 | 14 | COL1A1 | 2221.97 | ADGQPGAKGEPGDAGAKGDAGPPGP |
| 15 | 15 | COL1A1 | 2205.99 | ADGQPGAKGEPGDAGAKGDAGPPGP |
| 16 | 16 | COL1A1 | 2277.01 | ADGQPGAKGEPGDAGAKGDAGPPGPA |
| 17 | 17 | COL1A1 | 2293.01 | ADGQPGAKGEPGDAGAKGDAGPPGPA |
| 18 | 18 | COL1A1 | 2617.15 | GPPGADGQPGAKGEPGDAGAKGDAGPPGPA |
| 19 | 19 | COL1A1 | 2086.93 | EGSPGRDGSPGAKGDRGETGPA |
| 20 | 20 | COL1A1 | 2157.96 | AEGSPGRDGSPGAKGDRGETGPA |
| 21 | 21 | COL1A1 | 3014.41 | ESGREGAPGAEGSPGRDGSPGAKGDRGETGPA |
| 22 | 22 | COL1A1 | 1266.58 | SPGPDGKTGPPGPA |
| 23 | 23 | COL1A1 | 2129.99 | DGKTGPPGPAGQDGRPGPPGPPG |
| 24 | 24 | COL1A1 | 2017.93 | GRPGEVGPPGPPGPAGEKGSPG |
| 25 | 25 | [symbol] | 2081.94 | DGPPGRDGQPGHKGERGYPG |
| 26 | 26 | [symbol] | 2195.99 | NDGPPGRDGQPGHKGERGYPG |

The peptides above have been grouped into overlapping sets (by line breaks) and
aligned accordingly (i.e., 1-3, 4-5, 6-11, 12-13, 14-18, 19-21, 22-23, and 25-26)

| | | | | |
|---|---|---|---|---|
| 27 | 27 | COL2A1 | 1861.85 | SNGNPGPPGPPGPSGKDGPK |
| 28 | 28 | [symbol] | 1738.76 | NDGAPGKNGERGGPGGPGP |
| 29 | 29 | [symbol] | 2008.93 | DGESGRPGRPGERGLPGPPG |
| 30 | 30 | [symbol] | 2079.92 | DAGAPGAPGGKGDAGAPGERGPPG |
| 31 | 31 | [symbol] | 2565.18 | GAPGQNGEPGGKGERGAPGEKGEGGPPG |
| 32 | 32 | [symbol] | 2743.24 | KNGETGPQGPPGPTGPGGDKGDTGPPGPQG |
| 33 | 33 | COL4A1 | 1424.66 | PGQQGNPGAQGLPGP |
| 34 | 34 | COL4A2 | 1126.51 | GLPGLPGPKGFA |
| 35 | 35 | COL4A3 | 1161.52 | GEPGPPGPPGNLG |
| 36 | 36 | COL4A4 | 1218.55 | GLPGPPGPKGPRG |
| 37 | 37 | COL4A5 | 1144.52 | GPPGPPGPLGPLG |
| 38 | 38 | COL4A5 | 1269.53 | PGLDGMKGDPGLP |
| 39 | 39 | COL4A5 | 1733.76 | GIKGEKGNPGQPGLPGLP |
| 40 | 40 | COL4A6 | 1158.52 | GLPGPPGPPGPPS |
| 41 | 41 | COL5A1 | 1748.82 | KGPQGKPGLAGMPGANGPP |
| 42 | 42 | COL7A1 | 1690.80 | PGLPGQVGETGKPGAPGR |
| 43 | 43 | COL9A1 | 1732.84 | KRPDSGATGLPGRPGPPG |
| 44 | 44 | COL11A1 | 1441.64 | GPPGPPGLPGPQGPKG |
| 45 | 45 | COL11A1 | 1828.84 | DGPPGPPGERGPQGPQGPV |
| 46 | 46 | COL17A1 | 1368.62 | LPGPPGPPGSFLSN |
| 47 | 47 | COL18A1 | 1142.51 | GPPGPPGPPGPPS |

“P” residues in bold underline are hydroxyproline “D or N or Q” residues in bold underline are deaminated D or N or Q.
Genes labeled in bold italics were found to be significantly regulated in biopsy tissues in microarray data (see below).
M/Z: MALDI data analyzed by an algorithm that looks for sites (m/z values) whose intensity is higher the estimated average background and the ~100 surrounding sites, with peak widths ~0.5% of the corresponding m/z value. Peptides with underlined M/Z values are part of the 53 biomarker panel.

TABLE 1B
Uromodulin-derived (UMOD) Peptides

| | SEQ | | | |
|---|---|---|---|---|
| | ID | Precursor | | |
| S.No | NO: | Gene | M/Z | Peptide Sequence |
| 1 | 48 | [symbol] | 982.59 | VLNLGPITR |
| 2 | 49 | [symbol] | 1047.48 | SGSVIDQSRV |
| 3 | 50 | [symbol] | 1211.66 | DQSRVLNLGPI |
| 4 | 51 | [symbol] | 1225.69 | SRVLNLGPITR |
| 5 | 52 | [symbol] | 1324.76 | IDQSRVLNLGPI |
| 6 | 53 | [symbol] | 1423.83 | VIDQSRVLNLGPI |
| 7 | 54 | [symbol] | 1468.82 | DQSRVLNLGPITR |
| 8 | 55 | [symbol] | 1510.87 | SVIDQSRVLNLGPI |
| 9 | 56 | [symbol] | 1567.91 | GSVIDQSRVLNLGPI |
| 10 | 57 | [symbol] | 1581.91 | IDQSRVLNLGPITR |
| 11 | 58 | [symbol] | 1654.91 | SGSVIDQSRVLNLGPI |
| 12 | 59 | [symbol] | 1680.98 | VIDQSRVLNLGPITR |
| 13 | 60 | [symbol] | 1755.96 | SGSVIDQSRVLNLGPIT |
| 14 | 61 | [symbol] | 1768.01 | SVIDQSRVLNLGPITR |
| 15 | 62 | [symbol] | 1912.07 | SGSVIDQSRVLNLGPITR |
| 16 | 63 | [symbol] | 2040.16 | SGSVIDQSRVLNLGPITRK |

UMOD (in bold italics) was found to be significantly regulated in biopsy tissues in microarray data.

The UMOD peptide biomarker cluster discovered in this study spans from serine residue 589 (S⁵⁸⁹), following arginine residue 588 (R⁵⁸⁸), and to 607 residue lysine (K⁶⁰⁷) (Table 1C).

TABLE 1C
Uromodulin Amino Acid Sequence

SEQUENCE: 640 AMINO ACIDS
MW: 69761
SEQ ID NO: 64
001 MGQPSLTWML MVVVASWFIT TAATDTSEAR WCSECHSNAT CTEDEAVTTC TCQEGFTGDG
061 LTCVDLDECA IPGAHNCSAN SSCVNTPGSF SCVCPEGFRL SPGLGCTDVD ECAEPGLSHC
121 HALATCVNVV GSYLCVCPAG YRGDGWHCEC SPGSCGPGLD CVPEGDALVC ADPCQAHRTL
181 DEYWRSTEYG EGYACDTDLR GWYRFVGQGG ARMAETCVPV LRCNTAAPMW LNGTHPSSDE
241 GIVSRKACAH WSGHCCLWDA SVQVKACAGG YYVYNLTAPP ECHLAYCTDP SSVEGTCEEC
301 SIDEDCKSNN GRWHCQCKQD FNITDISLLE HRLECGANDM KVSLGKCQLK SLGFDKVFMY
361 LSDSRCSGFN DRDNRDWVSV VTPARDGPCG TVLTRNETHA TYSNTLYLAD EIIIRDLNIK
421 INFACSYPLD MKVSLKTALQ PMVSALNIRV GGTGMFTVRM ALFQTPSYTQ PYQGSSVTLS
481 TEAFLYVGTM LDGGDLSRFA LLMTNCYATP SSNATDPLKY FIIQDRCPHT RDSTIQVVEN
541 GESSQGRFSV QMFRFAGNYD LVYLHCEVYL CDTMNEKCKP TCSGTRFRSG SVIDQSRVLN
601 LGPITRKGVQ ATVSRAFSSL GLLKVWLPLL LSATLTLTFQ
The UMOD peptide biomarker cluster discovered in this study spans from serine residue 589 (S⁵⁸⁹) to lysine residue 607 (K⁶⁰⁷; double underlined sequence) which following arginine residue 588 (R⁵⁸⁸). Spectrometry analyses (ref. 47) has shown that C-terminal cleavage of the UMOD precursor, which has 640 amino acids, occurs after the phenylalanine residue 587 (F⁵⁸⁷; bold underline).

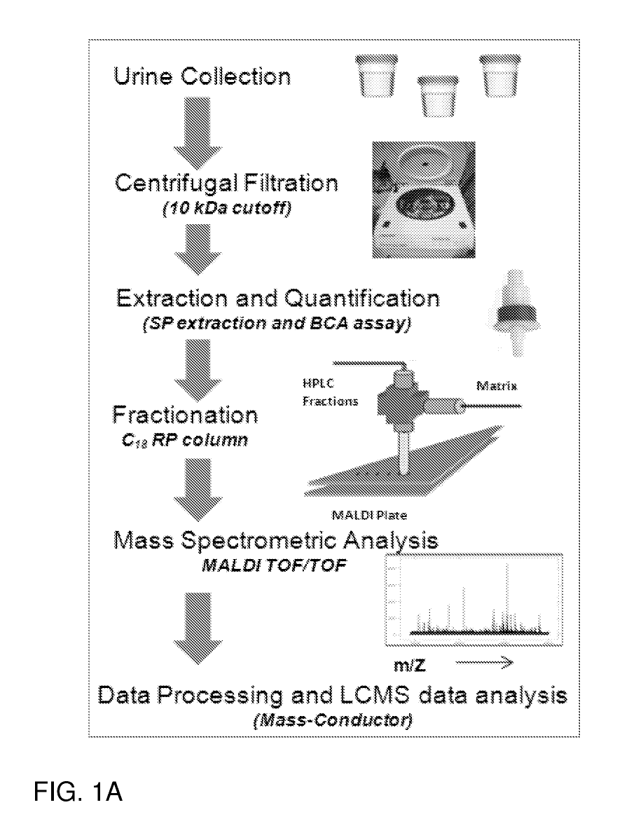

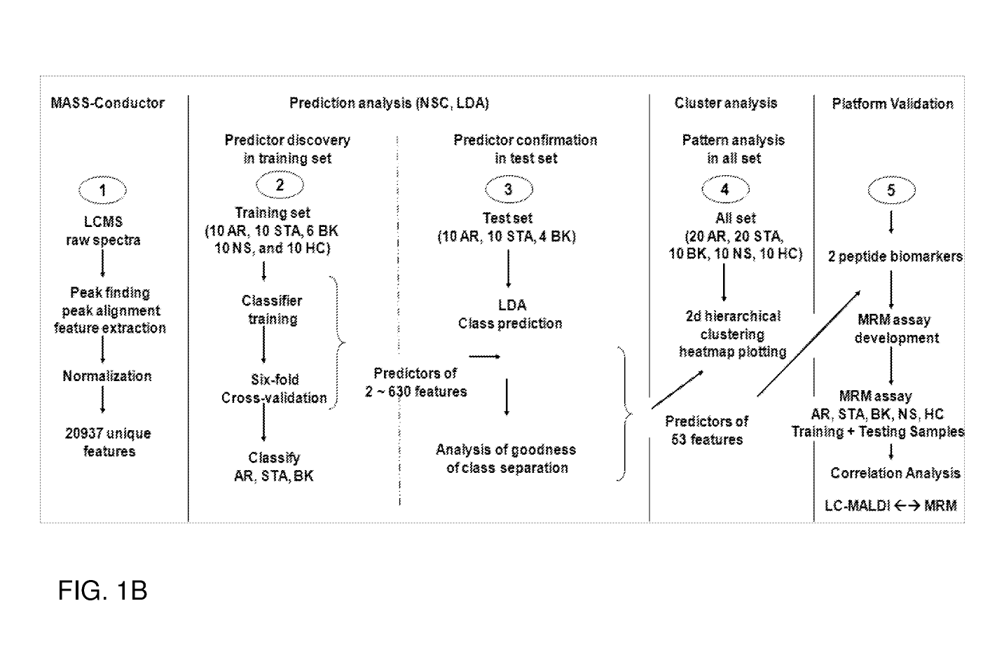

The peptide signature of a sample can be obtained using any convenient method for peptide analysis. As such, no limitation in this regard is intended. Exemplary peptide analysis includes, but is not limited to: HPLC, mass spectrometry, LC-MS based peptide profiling (e.g., LC-MALDI as shown in FIG. 1), Multiple Reaction Monitoring (MRM), ELISA, and the like.

In certain embodiments, a biopsy sample from the transplanted kidney is assayed to obtain a gene expression level evaluation, e.g., a gene expression profile, which is employed to determine a clinical transplant category of the subject who has received the transplanted kidney. In certain embodiments, the expression profile includes expression data for one or more genes selected from COL1A2, COL3A1, MMP7, SERPING1, TIMP1 and UMOD, where the term expression profile is used broadly to include a genomic expression profile, e.g., an expression profile of nucleic acid transcripts, e.g., mRNAs, of the one or more genes of interest, or a proteomic expression profile, e.g., an expression profile of one or more different proteins, where the proteins/polypeptides are expression products of the one or more genes of interest. As such, in certain embodiments the expression level of only one gene selected from COL1A2, COL3A1, MMP7, SERPING1, TIMP1 and UMOD is evaluated, e.g., COL1A2. In yet other embodiments, the expression level of two or more genes selected from COL1A2, COL3A1, MMP7, SERPING1, TIMP1 and UMOD is evaluated, e.g., 3, 4, 5 or all 6 genes. In certain embodiments, the expression level of one or more additional gene other than those selected from COL1A2, COL3A1, MMP7, SERPING1, TIMP1 and UMOD is also evaluated.

In certain embodiments, both a peptide signature, e.g., from a urine sample, and a gene expression profile, e.g., from a biopsy sample, is obtained for a subject having a kidney transplant, both of which are employed to determine a transplant category of the subject.

In the broadest sense, peptide or gene expression evaluation may be qualitative or quantitative. As such, where detection is qualitative, the methods provide a reading or evaluation, e.g., assessment, of whether or not the target analyte, e.g., peptide, nucleic acid or other expression product (e.g., protein), is present in the sample being assayed. In yet other embodiments, the methods provide a quantitative detection of whether the target analyte is present in the sample being assayed, i.e., an evaluation or assessment of the actual amount or relative abundance of the target analyte, e.g., peptide or nucleic acid in the sample being assayed. In such embodiments, the quantitative detection may be absolute or, if the method is a method of detecting two or more different analytes in a sample, relative. As such, the term “quantifying” when used in the context of quantifying a target analyte in a sample can refer to absolute or to relative quantification. Absolute quantification may be accomplished by inclusion of known concentration(s) of one or more control analytes and referencing the detected level of the target analyte(s) with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other. In addition, a relative quantitation may be ascertained using a control, or reference, sample (e.g., as is commonly done in array based assays as well as in quantitative PCR/RT-PCR analyses or sequencing and analysis of the transcriptome).

In certain embodiments, additional analytes beyond those listed above may be assayed. For example, for biopsy samples, other genes whose expression level/pattern is modulated under different transplant conditions (e.g., during an AR response) can be evaluated. In certain embodiments, a non-biopsy sample can be evaluated to obtain a gene expression result (e.g., from blood or blood derived cells) that can be used to evaluate additional transplant characteristics, including but not limited to: a graft tolerant phenotype in a subject, chronic allograft injury (chronic rejection); immunosuppressive drug toxicity or adverse side effects including drug-induced hypertension; age or body mass index associated genes that correlate with renal pathology or account for differences in recipient age-related graft acceptance; immune tolerance markers; genes found in literature surveys with immune modulatory roles that may play a role in transplant outcomes. In addition, other function-related genes may be evaluated, e.g., for assessing sample quality (3′- to 5′-bias in probe location), sampling error in biopsy-based studies, cell surface markers, and normalizing genes for calibrating hybridization results (exemplary genes in these categories can be found in U.S. patent application Ser. No. 11/375,681, filed on Mar. 3, 2006, which is incorporated by reference herein in its entirety).

In certain embodiments, additional genes are evaluated to determine whether a subject who has received an allograft has a graft tolerant phenotype, e.g., as described in provisional patent application 61/089,805, filed on Aug. 18, 2008, which is incorporated herein by reference in its entirety. By graft tolerant phenotype is meant that the subject does not reject a graft organ, tissue or cell(s) that has been introduced into/onto the subject. In other words, the subject tolerates or maintains the organ, tissue or cell(s) that has been transplanted to it. A feature of the graft tolerant phenotype detected or identified is that it is a phenotype which occurs without immunosuppressive therapy, i.e., it is present in a subject that is not undergoing immunosuppressive therapy such that immunosuppressive agents are not being administered to the host.

In certain embodiments, the expression profile obtained is a genomic or nucleic acid expression profile, where the amount or level of one or more nucleic acids in the sample is determined, e.g., the nucleic acid transcript of the gene of interest. In these embodiments, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. In certain embodiments, the sample is prepared from a cell or tissue harvested from a subject to be diagnosed, e.g., via biopsy of tissue, using standard protocols, where cell types or tissues from which such nucleic acids may be generated include any tissue in which the expression pattern of the to be determined phenotype exists, including, but not limited to, peripheral blood lymphocyte cells, etc., as reviewed above.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. In certain embodiments, such applications are hybridization assays in which a nucleic acid array that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the levels of one or more nucleic acids in a sample may be employed, including quantitative PCR, real-time quantitative PCR, and the like. (For general details concerning real-time PCR see Real-Time PCR: An Essential Guide, K. Edwards et al., eds., Horizon Bioscience, Norwich, U.K. (2004)).

Where the expression profile is a protein expression profile, any convenient protein quantitation protocol may be employed, where the levels of one or more proteins in the assayed sample are determined. Representative methods include, but are not limited to: MRM analysis, standard immunoassays (e.g., ELISA assays), protein activity assays, including multiplex protein activity assays, etc. Following obtainment of the peptide signature and/or gene expression data, or gene expression profile (or signature), from a subject, the peptide signature and/or gene expression signature is analyzed. In certain embodiments, analysis includes comparing the peptide signature and/or gene expression signature with a reference or control signature to determine the transplant category of the transplant subject. The terms “reference” and “control” as used herein mean a standardized analyte level (or pattern) that can be used to interpret the analyte pattern of a sample from a subject. The reference or control profile may be a profile that is obtained from a subject having an AR phenotype, and therefore may be a positive reference or control signature for AR. In addition, the reference/control profile may be from a subject known to not be undergoing AR (e.g., STA, BK, NS or HC), and therefore be a negative reference/control signature.

In certain embodiments, the obtained peptide signature and/or gene expression profile is compared to a single reference/control profile to obtain information regarding the subject's transplant category. In yet other embodiments, the obtained peptide signature and/or gene expression profile is compared to two or more different reference/control profiles to obtain more in depth information regarding the transplant category of the subject. For example, the obtained peptide signature and/or gene expression profile may be compared to a positive and negative reference profile to obtain confirmed information regarding whether the subject is undergoing an AR response.

The comparison of the obtained peptide signature and/or gene expression profile and the one or more reference/control profiles may be performed using any convenient methodology, where a variety of methodologies are known to those of skill in the array art, e.g., by comparing digital images of the peptide signatures and/or gene expression profiles, by comparing databases of peptide signatures and/or gene expression profiles, etc. Patents describing ways of comparing expression profiles include, but are not limited to, U.S. Pat. Nos. 6,308,170 and 6,228,575, the disclosures of which are herein incorporated by reference.

The comparison step results in information regarding how similar or dissimilar the obtained peptide signature and/or gene expression profile is to the control/reference profile(s), which similarity/dissimilarity information is employed to determine the transplant category of the subject. For example, similarity of the obtained peptide signature and/or gene expression profile with the peptide signature and/or gene expression profile of a control sample from a subject experiencing an active AR response indicates that the subject is experiencing AR. Likewise, similarity of the obtained peptide signature and/or gene expression profile with the peptide signature and/or gene expression profile of a control sample from a subject that has not had (or isn't having) an AR episode (e.g., STA) indicates that the subject is not experiencing AR.

Depending on the type and nature of the reference/control profile(s) to which the obtained peptide signature and/or gene expression profile is compared, the above comparison step yields a variety of different types of information regarding the subject as well as the sample employed for the assay. As such, the above comparison step can yield a positive/negative determination of an ongoing AR response. In certain embodiments, the determination/prediction of AR can be coupled with a determination of additional characteristics of the graft and function thereof. For example, in certain embodiments one can assay for other graft-related pathologies, e.g., chronic rejection (or CAN) and/or drug toxicity (DT) (see, e.g., U.S. patent application Ser. No. 11/375,681, filed on Mar. 3, 2006, which is incorporated by reference herein in its entirety).

The subject methods further find use in pharmacogenomic applications. In these applications, a subject/host/patient is first monitored for their clinical transplant category (e.g., for an AR response) according to the subject invention, and then treated using a protocol determined, at least in part, on the results of the monitoring. For example, a host may be evaluated for the presence or absence of AR using a protocol such as the diagnostic protocol described above. The subject may then be treated using a protocol whose suitability is determined using the results of the monitoring step. For example, where the subject is categorized as having an AR response, immunosuppressive therapy can be modulated, e.g., increased or drugs changed, as is known in the art for the treatment/prevention of AR. Likewise, where the subject is categorized as free of AR, the immunosuppressive therapy can be reduced, e.g., in order to reduce the potential for DT.

In practicing the subject methods, a subject is typically monitored for AR following receipt of a graft or transplant. The subject may be screened once or serially following transplant receipt, e.g., weekly, monthly, bimonthly, half-yearly, yearly, etc. In certain embodiments, the subject is monitored prior to the occurrence of an AR episode. In certain other embodiments, the subject is monitored following the occurrence of an AR episode.

The subject methods may be employed with a variety of different types of transplant subjects. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) are humans.

### Databases of Expression Profiles of Phenotype Determinative Genes

Also provided are databases of peptide signatures and/or gene expression profiles of different transplant categories, e.g., AR, STA, NS, BK and the like. The peptide signatures and/or gene expression profiles and databases thereof may be provided in a variety of media to facilitate their use (e.g., in a user-accessible/readable format). “Media” refers to a manufacture that contains the expression profile information of the present invention. The databases of the present invention can be recorded on computer readable media, e.g. any medium that can be read and accessed directly by a user employing a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. Thus, the subject expression profile databases are accessible by a user, i.e., the database files are saved in a user-readable format (e.g., a computer readable format, where a user controls the computer).

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention, e.g., to and from a user. One format for an output means ranks expression profiles possessing varying degrees of similarity to a reference expression profile. Such presentation provides a skilled artisan (or user) with a ranking of similarities and identifies the degree of similarity contained in the test expression profile to one or more references profile(s).

As such, the subject invention further includes a computer program product for determining a clinical transplant category of a subject who has received a kidney allograft. The computer program product, when loaded onto a computer, is configured to employ a peptide signature from a non-invasive sample and/or a gene expression signature from a biopsy sample from said subject to determine a clinical transplant category for the subject. Once determined, the clinical transplant category is provided to a user in a user-readable format. In certain embodiments, the peptide signature includes data for the peptide level of one or more peptides listed in SEQ ID NOs: 1 to 63. A gene expression signature includes gene expression level data for one or more genes COL1A2, COL3A1, MMP7, SERPING1, TIMP1 and UMOD. In addition, the computer program product may include one or more reference or control peptide and/or gene expression signatures (as described in detail above) which are employed to determine the clinical transplant category of the patient.

### Reagents, Systems and Kits

Also provided are reagents, systems and kits thereof for practicing one or more of the above-described methods. The subject reagents, systems and kits thereof may vary greatly. Reagents of interest include reagents specifically designed for use in production of the above-described peptide signatures and/or gene expression profiles. These include a peptide level or gene expression evaluation element made up of one or more reagents. The term system refers to a collection of reagents, however compiled, e.g., by purchasing the collection of reagents from the same or different sources. The term kit refers to a collection of reagents provided, e.g., sold, together.

One type of such reagent is an array of probe nucleic acids in which the phenotype determinative genes of interest are represented, i.e., COL1A2, COL3A1, MMP7, SERPING1, TIMP1 and/or UMOD. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Probes for any combination of genes listed above may be employed. The subject arrays may include only those genes that are listed above or they may include additional genes that are not listed above, such as probes for genes whose expression pattern can be used to evaluate additional transplant characteristics as well as other array assay function related genes, e.g., for assessing sample quality (3′- to 5′-bias in probe location), sampling error in biopsy-based studies, cell surface markers, and normalizing genes for calibrating hybridization results; and the like. Transplant characterization genes are genes whose expression can be employed to characterize transplant function in some manner, e.g., presence of rejection, etc.

Another type of reagent that is specifically tailored for generating expression profiles of phenotype determinative genes is a collection of gene specific primers that is designed to selectively amplify such genes (e.g., using a PCR-based technique, e.g., real-time RT-PCR). Gene specific primers and methods for using the same are described in U.S. Pat. No. 5,994,076, the disclosure of which is herein incorporated by reference. Of particular interest are collections of gene specific primers that have primers for at least 1 of the genes selected from COL1A2, COL3A1, MMP7, SERPING1, TIMP1 and UMOD, often a plurality of these genes, e.g., at least 2, 3, 4, 5 or all 6 genes. The subject gene specific primer collections may include primers specific for only those genes listed above, or they may include primers for additional genes, such as probes for genes whose expression pattern can be used to evaluate additional transplant characteristics as well as other array assay function related genes, as noted above.

The systems and kits of the subject invention may include the above-described arrays and/or gene specific primer collections. The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

The subject systems and kits may further include reagents for peptide or protein level determination, for example those that find use in ELISA assays, Western blot assays, MS assays (e.g., LC-MS), HPLC assays, flow cytometry assays, and the like.

The subject systems and kits may also include a phenotype determination element, which element is, in many embodiments, a reference or control peptide signature or gene expression profile that can be employed, e.g., by a suitable computing means, to determine a transplant category based on an “input” peptide signature and/or gene expression profile. Representative phenotype determination elements include databases of peptide signatures or gene expression profiles, e.g., reference or control profiles, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### Introduction

Despite an improvement in renal allograft survival reflecting advances in immunosuppressive medications (1,2), an unmet need in patient care is the requirement for sensitive and graft etiology specific, non invasive methodologies for monitoring transplant recipients (3). Expression analyses of urine immune mediators (4), peripheral blood samples and transplant biopsies (5,6) support that distinct molecular pathways can define the injury of acute rejection (AR). Some of the concerns relating to biomarker discovery in urine lie with the confounding effect of proteinuria and high abundance plasma proteins from non-specific injury (which also occurs in AR). In this study, we analyze naturally occurring peptides in urine samples from transplant patients. Reasons for analyzing naturally occurring peptides in urine include: 1) As the roughly equal mass of protein and peptide in urine translates into at least a ten-fold greater molar abundance of peptides, urinary peptides provide a fertile ground for biomarker discovery; 2) Urinary peptide analysis, unlike intact urinary proteomics analysis, is not hampered by the presence of highly abundant urinary proteins that can obscure the discovery of more informative lower abundance biomarker proteins (7); and 3) analysis of urinary peptides is relatively easier than the analysis of complex tissues such as biopsy and blood as one dimensional HPLC separation is sufficient for the analysis of greater than 25,000 urine peptides (7).

One confounder for AR diagnosis and management is BK nephritis. To address these issues, this study performs non-invasive, urine peptidomic analysis of 70 unique urine samples, collected form renal transplant patients and controls, by liquid chromatography and mass spectrometry (LC-MS), followed by MRM verification, on 5 different cohorts, including samples with non-specific proteinuria and BK nephritis and vyuria.

We also performed integrated transcriptomic analysis on matching biopsy microarrays, paired with the urine samples, available in the lab of Dr. Sarwal (GEO, GSE14328). Significant overlapping genes were verified by quantitative real time PCR (Q-PCR) in an independent set of 34 biopsy samples.

Our results indicate that disease specific alteration of proteolytic and anti-proteolytic activities is the underlying mechanism by which these urine peptide biomarkers are generated during graft rejection. To our knowledge, this study represents the first study which analyzed both urinary peptidomic and matching renal biopsy transcriptomic analyses, which will help in elucidating the pathophysiological relationships between our nested urine peptide biomarkers and allograft proteolytic networks in vivo in renal allograft diseases.

### Results

### Sample Characteristics

The overall study design for the peptidomic urine analysis is shown in FIGS. 1A and B. Seventy unique urine samples were analyzed from the following 5 cohorts: pediatric kidney transplant patients with biopsy proven acute allograft rejection (AR, n=20), stable allograft with normal protocol biopsies (STA, n=20), BK virus nephropathy with vyurina (BK, n=10), non-specific proteinuria with native renal disease (biopsy proven nephrotic syndrome) (NS, n=10) and healthy age matched volunteers (HC, n=10). Samples were split into Training Sets (n=46) for urine peptide discovery, and Test Sets (n=24) (sample demographics in Table 2) for urine peptide prediction and verification.

### Discovery of a Urine Peptide Panel for AR by LC-MALDI

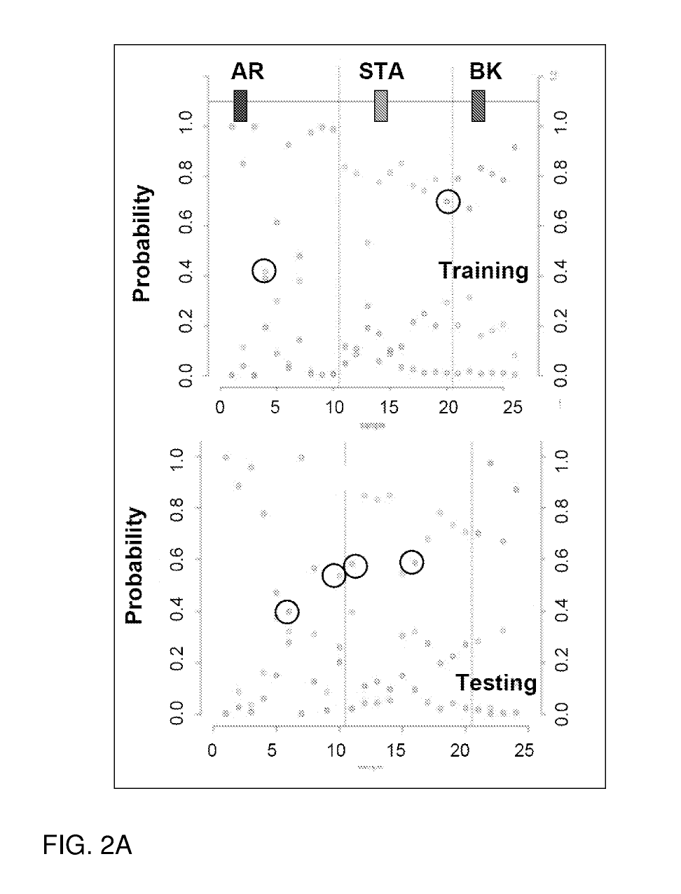

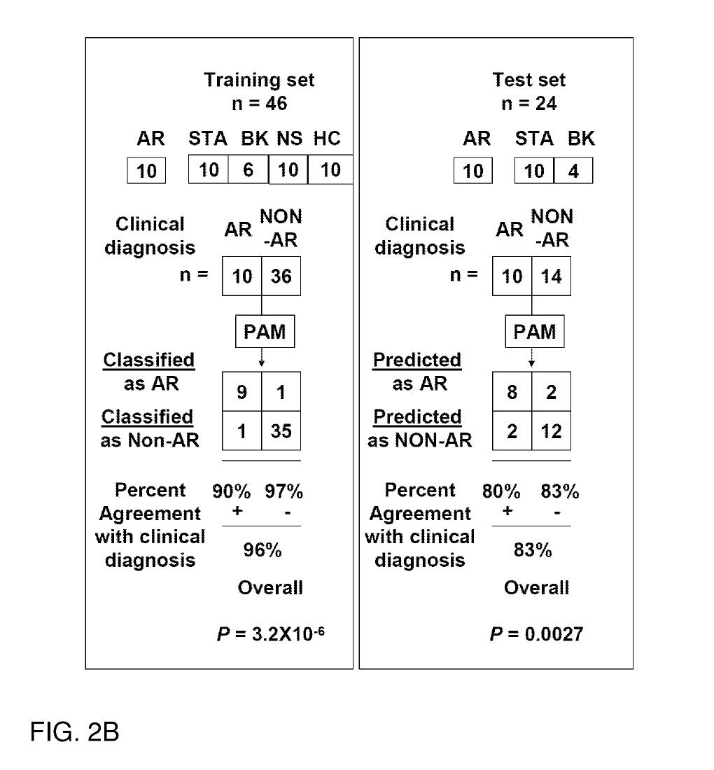

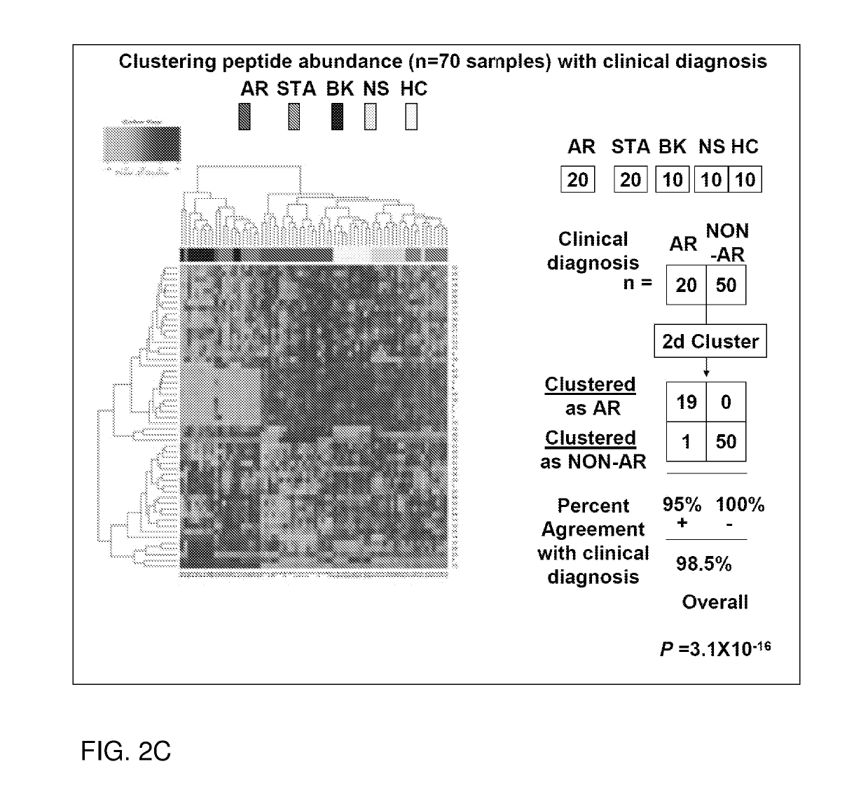

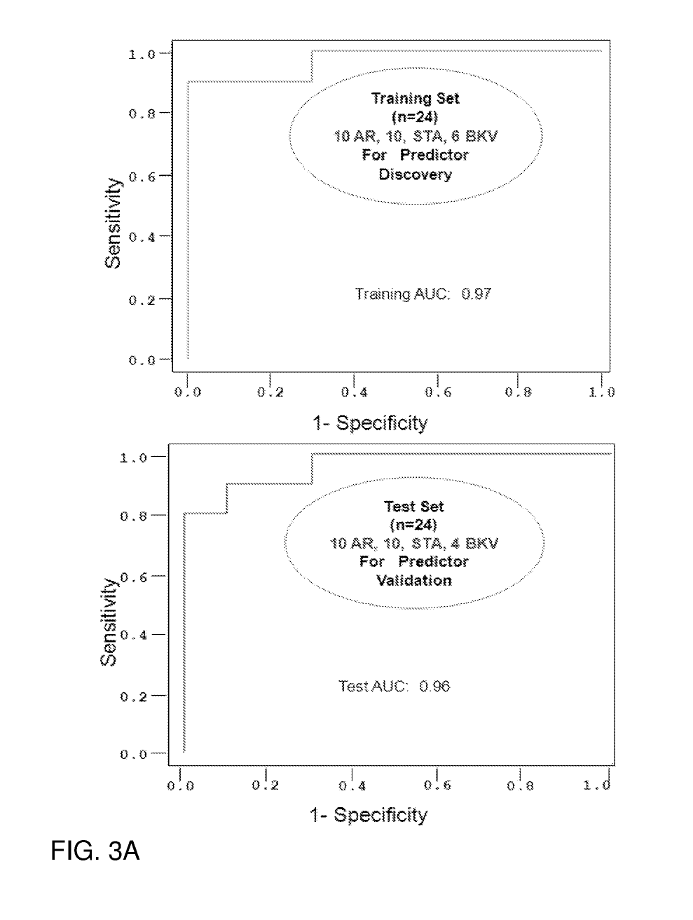

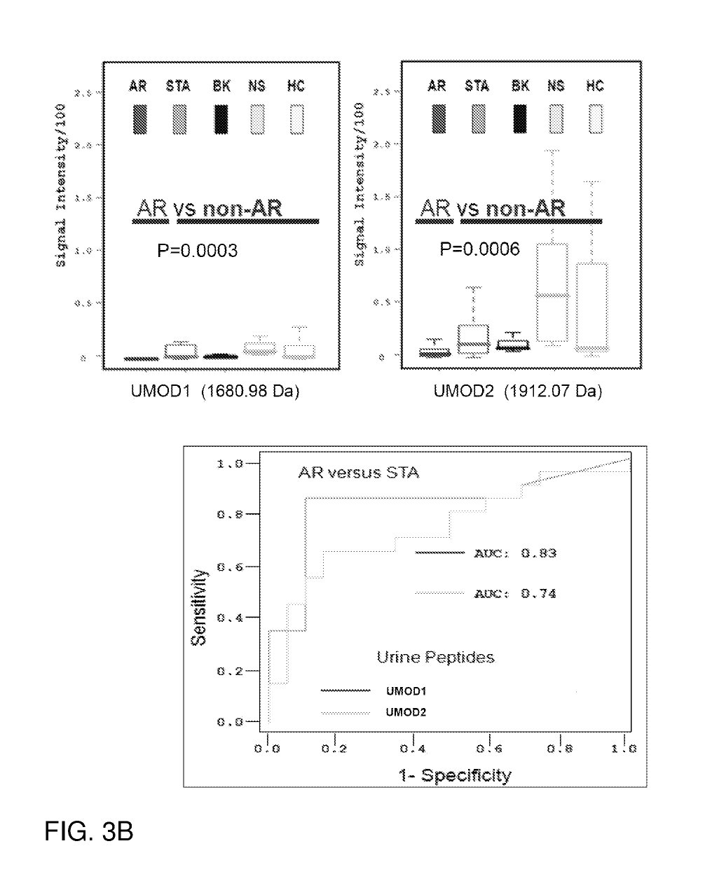

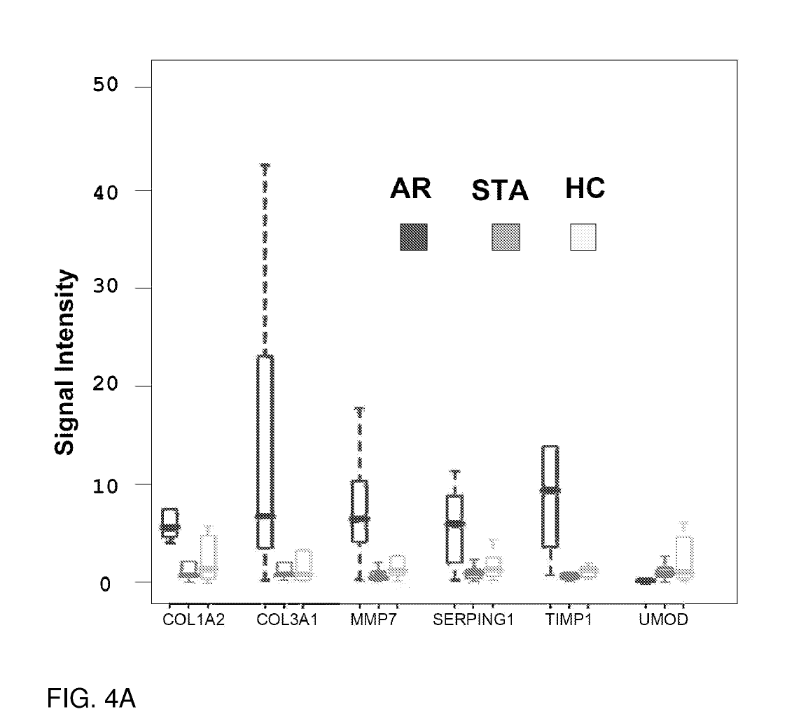

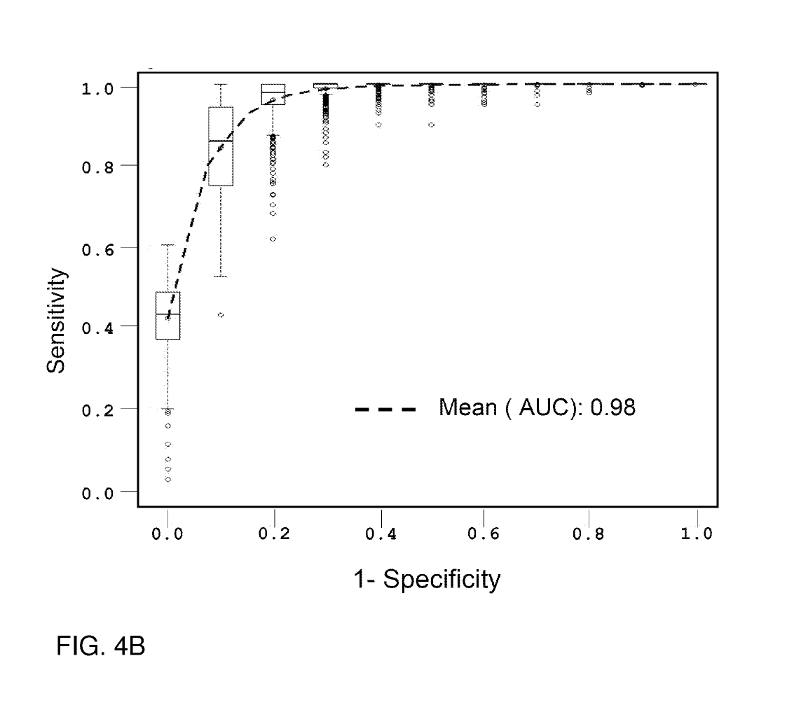

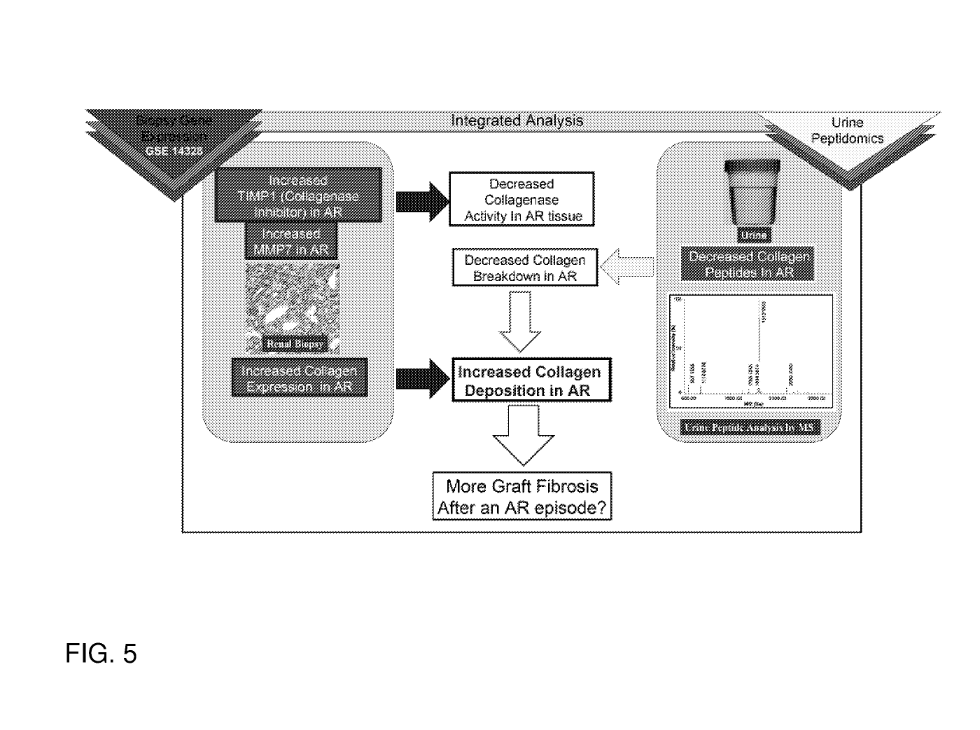

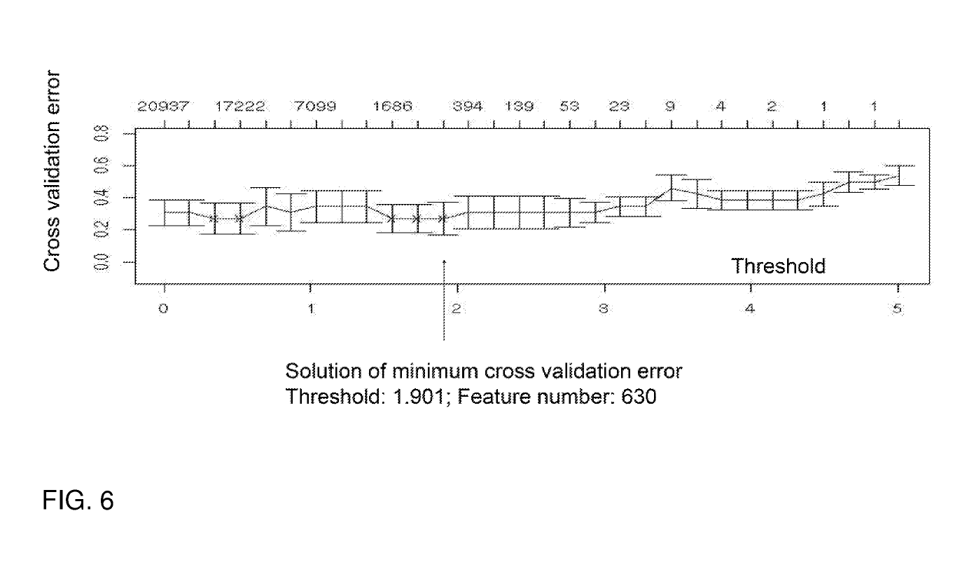

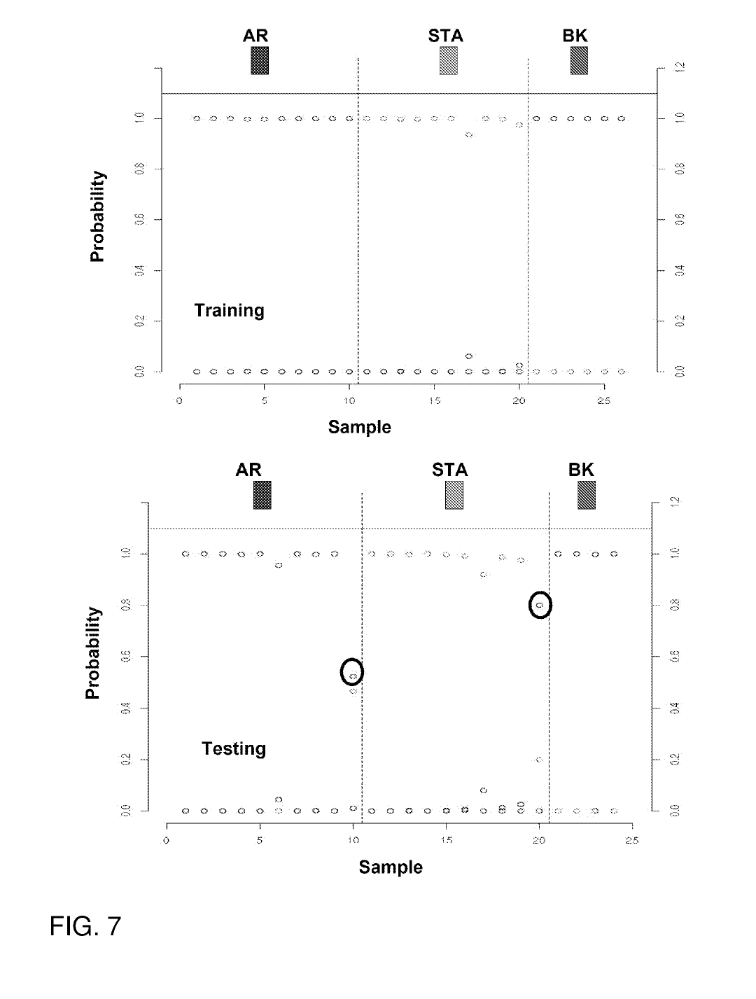

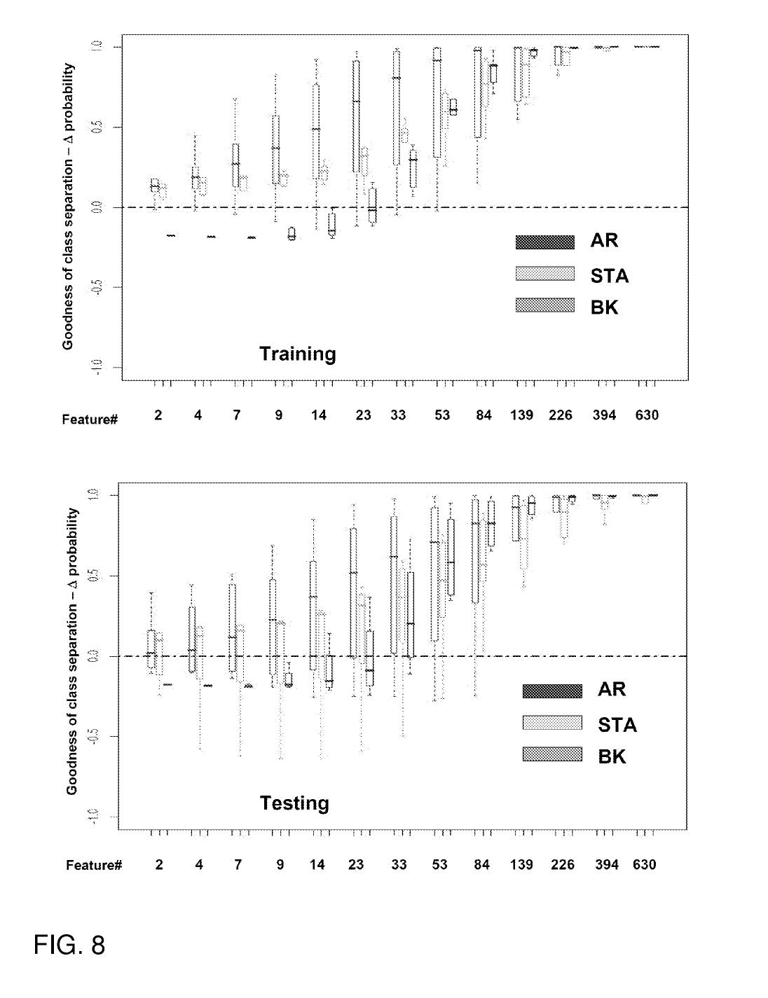

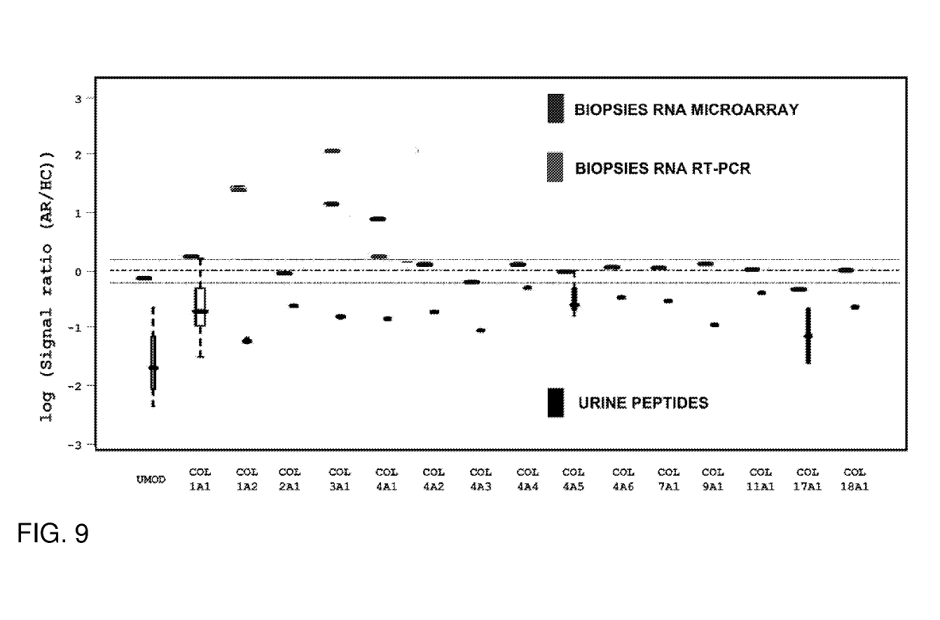

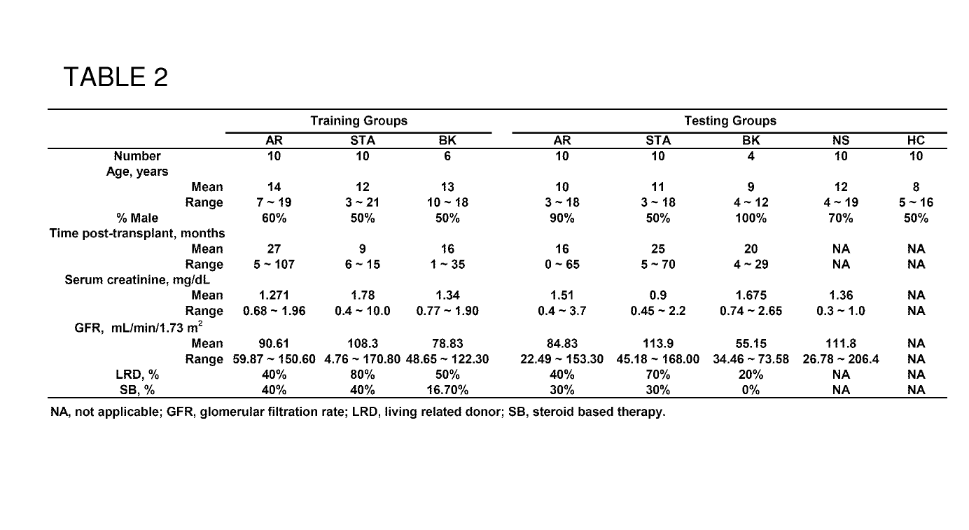

A total of 20,937 unique peptide peaks with distinct m/z and HPLC fractions were resolved in the 900 to 4000 Da range. Prediction analysis by a nearest shrunken centroid (NSC) algorithm (8) was performed and 10-fold internal cross validation analysis led to the discovery of a set of 630 peptide features (FIG. 6). Discriminant class probabilities and Gaussian linear discriminant analysis (LDA) were performed for each sample (8) (FIG. 7) in both sample sets, and resulted in misclassification of only 2 of the 24 samples in the test set. To find a predictive biomarker panel of optimal feature number, various classifiers were tested for their spread of distribution and of the goodness of the separation (FIG. 1B and FIG. 8). Linear discriminant probabilities of a 53 peptide biomarker panel was sufficient for goodness of separation of the clinically relevant transplant categories (AR, STA and BK) in both the training and the test sample sets (FIGS. 2A and 2B) The high resolution mass spectrometric analysis uncovered 53 mass spectrometric peaks discriminating different allograft dysfunction classes. Subsequent deconvoluting and deisotoping analysis found 40 unique peptides from these 53 peaks, upon which a mathematic model was developed as a classifier to discriminate different allograft dysfunctions (AR, STA and BK). Urine naturally occurring peptide catalog analysis found that different overlapping peptides (total of 63 peptides, Table 1A and 1B) cluster with differential disease predictive power. The 53 peptide biomarker panel classified the AR samples with 96% agreement with clinical diagnosis of AR in the training set (p=3.2×10⁻⁶ by Fisher exact test) and 83% agreement with clinical diagnosis of AR in the test set (p=of 0.0027 by Fisher exact test). When all 70 samples were clustered by unsupervised analysis of their peptide abundance across the 53 peak features, all AR samples, save one, co-clustered, and importantly, all the non-AR samples (STA, BK, NS and HC) clustered disparate form the AR sample cluster (FIG. 2C). Interestingly, the STA samples separated into 2 clusters suggesting that STA samples might harbor two subclasses at the urine peptide level. Based upon the discriminant analysis derived prediction scores for each sample, a receiver operating characteristic (ROC) curve was constructed using all 53 peptides (9, 10) and resulted in area under the curve (AUC) values of 0.97 and 0.96 for the training and the test-set respectively (FIG. 3A).

### Identification of AR-Specific Urine Peptides

Manual review of the biomarker panel and associated MS spectra interpreted and de-isotoped the 53 MS peak features, which could be mapped to 40 unique urine peptides, and were further identified by MALDI-TOF/TOF and LTQ Orbitrap MS/MS analysis. We grouped the identified peptides according to their common protein precursor and computed the medians of LC-MS measurements according to sample categories. The peptides were found to map to 9 different proteins, 8 of which belonged to the collagen family (COL1A1, COL1A2, COL3A1, COL4A3, COL4A4, COL4A5, COL7A1, COL18A1) and UMOD. When MS/MS analysis was extended to the original 630 peptide feature set, 142 urine peptides were identified, again with predominant presence of collagen peptides (n=47) and UMOD peptides (n=16) (Table 1A, 1B). The UMOD peptide biomarker cluster discovered in this study spans from serine residue 589 (S⁵⁸⁹), following arginine residue 588 (R⁵⁸⁸), and to 607 residue lysine (K⁶⁰⁷) (Table 1C). Little is known about the metabolic pathway of this C-terminal peptide and its biological role after UMOD is shed from the apical plasma membrane into the tubule lumen. Uromodulin (UMOD), the most abundant urinary protein in mammals, has been recently shown to be significantly lower in abundance in urine samples from patients with renal transplant rejection (11). UMOD peptides analyzed in pooled urine samples have also been found to be significantly reduced in patients with transplant rejection, compared to patients without rejection (7). This study confirms the results that UMOD peptides are much lower in individual urine samples taken from patients when the filtering kidney has ongoing acute rejection. Though the significance of these findings is unclear at present, a recent genome wide association study has identified significant SNP associations with chronic kidney disease at the UMOD locus (12).

Interestingly, all of the identified urine peptides showed much lower abundance during AR when compared to other samples, with overall lower abundance in transplant patients, when compared to non-transplanted patients (NS) and healthy controls (FIG. 9). Sequence alignment analysis of the collagen and UMOD peptides were found to line up by forming clusters within either the C or N terminal end with ladder like truncations at the opposite ends, suggesting that there is likely proteolytic degradation of the parent protein. Similar to the proteolytic degradation of urine proteins in AR, serum proteins have also been found to show differences in degradation in cancer (13).

### MRM Verification of Selected Urine Peptides

To verify the presence and quantify differences in peptides between AR and non-AR groups, MRM were performed on 2 selected peptides; (14) UMOD1 1680.98 Da and UMOD2 1912.07 Da FIG. 3) on all 70 samples. The box-whisker graphs in FIG. 3B illustrate the spread of the distribution of the MRM measurements in AR (n=20), STA (n=20), BK (n=10), NS (n=10), HC (n=10) sample categories for peptides with m/z 1680.98 Da and 1912.07 Da respectively. As seen in FIG. 3B (upper panel—left hand side), similar to the results obtained by LC-MALDI, the abundance of UMOD peptide 1680 was significantly lower in AR (p=0.0003), and as seen in FIG. 3B (upper panel—right hand side), the abundance of UMOD 1912 was also significantly lower in AR (p=0.0006), when compared to all other non-AR categories. ROC analysis to test the diagnostic ability of the two UMOD peptide biomarkers for AR was seen in terms of AUC. AUCs for UMOD1 and UMOD2 were 0.83 and 0.74 respectively.

### Integrated Analysis of Matched Samples: Transcriptional Analysis of Biopsy AR and Peptidomic Analysis of Urine AR

As urine is an ultrafiltrate of the kidney, we hypothesized that the alteration of the urinary proteins and peptides in urine, may relate to processes occurring directly in the kidney. To address this we analyzed archived microarray data in the Sarwal Lab (GSE14328), on matched kidney biopsies (20 AR and 20 STA; taken at the time of urine collection, prior to any treatment intensification for AR) for expression differences between AR and STA samples for the corresponding UMOD and the COL genes. We also looked for any expression differences in extracellular matrix proteins in AR, as some of these have been previously demonstrated to be differentially expressed in AR (15). We observed that whereas UMOD gene expression in AR biopsy was significantly lower in AR (false discovery rate or FDR=0/02%; similar results to the low UMOD peptide abundance in AR urine), the three COL genes (COL1A2, FDR=0.18%; COL3A1, FDR=0.67%; COL4A1, FDR=1.82%) were upregulated in AR (different from low COL peptide abundance in AR urine). Gene expression for matrix metalloproteinase-7 (MMP-7; FDR=0.03%), tissue inhibitor of metalloproteinase 1 (TIMP1; FDR=24%), and the serpin peptidase inhibitor (SERPING1; FDR=33%) was higher in AR when compared to STA biopsies, though only MMP7 expression was significant.

We performed quantitative real-time (RT) PCR in biopsies from a separate set of 34 kidney biopsies (14 AR, 10 STA and 10 healthy kidney donor biopsies) for UMOD, the most significant COL genes in rejection, namely COL1A2 and COL3A1, as well as all MMP7, SERPING1 and TIMP1 (FIG. 4A). The Q-PCR results showed that the 6 genes had statistically significant expression differences in AR, with similar results between the microarray and Q-PCR; lower gene expression for UMOD in AR (p=0.011), and higher gene expression for COL1A2 (p=0.027), COL3A1 (p=0.013), MMP7 (p=0.013), SERPING1 (p=0.005), and TIMP1 (p=0.013) in AR, when compared to samples without AR (FIG. 4A). The importance of these pathways is underscored by the finding that linear discriminant analysis can use the gene expression values of the 6 genes in biopsy AR tissue (ROC curve value of 0.98; FIG. 4B) to also accurately classify a rejection episode, similar to the results obtained from analysis of the corresponding urine peptides (FIG. 4A and FIG. 3). Interestingly, irrespective of the confounder of BK, biopsy UMOD gene expression and urinary peptide abundance are significantly lower in AR, whereas biopsy collagen gene expression is significantly higher in AR, whereas COL peptide abundance in rejecting urine is significantly lower. The dysregulation of collagen expression in the rejecting graft and altered proteolysis of collagens in the urine, may provide a novel insight into the cascade of events that prime a graft for chronic injury and fibrosis after an acute rejection episode (FIG. 5).

### Discussion

Proteomic and peptidomic analysis of urine collected from healthy individuals (22 mg peptides in urine/day; 48) and patients with renal disease, have identified more than 1500 different proteins (11,16,17) and over 100,000 different peptide biomarkers (18) in health and disease (19). This is the first study of an integrated analysis of the urine peptidome and the biopsy transcriptome in graft rejection, which uncovers that overlapping key gene and peptide pathways can be jointly dysregulated in acute rejection. The resultant alterations in the abundance of selected genes and the peptide products of the corresponding proteins can highlight potential mechanisms of graft injury in rejection. Injury specific alterations of gene transcription in the tissue, both by array and by Q-PCR, and a change in the balance of proteolytic and anti-proteolytic activities in urine, appear to be important mechanisms resulting in an altered pattern of a specific panel of urinary peptides in acute rejection.

There are at least 28 different human collagens that represent ˜25% of the total protein content of mammals (20), but in the kidney, type I and III collagen are most abundant, while type IV collagen is a major component of basement membranes (21). The increase in the amino-terminal and carboxy-terminal propeptides from the procollagen of types I, III, and IV during collagen anabolism and later decrease in the collagen-derived urinary naturally occurring peptides during collagen catabolism, suggest that increased turnover of renal collagens (22-25) may be valuable biomarkers for non-invasive diagnosis of the rejection process in the kidney.

The up-regulation of extracellular matrix proteins (MMP7, SERPING1 and TIMP1) also support the hypothesis of tissue remodeling at the time of acute rejection. The observance of high MMP-7 expression in the kidney at the time of acute rejection has also been previously reported in chronic kidney rejection (26), human kidney aging (27) and a rat renal acute rejection model (28). MMP-7 is a collagenase-related connective-tissue-degrading metalloproteinase and plays a role in the breakdown of extracellular matrix in normal physiological processes, tissue remodeling during injury (29) and neutrophil influx to sites of injury (30).

SERPRING1 regulates leukocyte trafficking and complement (inactivating C1r, C1s, MASP2, and C3b proteases) (31), which is also locally regulated in the kidney during ischemia reperfusion injury. Similar to the finding in this study, SERPING1 has also been shown to be regulated in the graft during acute rejection (32).

Tissue specific inhibitors of metalloproteinases (TIMPs) are endogenous, specific inhibitors that bind and inhibit MMPs (33). TIMP-1 is a physiological inhibitor of the matrix-degrading enzymes, collagenases, genlatinase and stromelysin and plays a major role in the inhibition of matrix degradation. Up-regulation of TIMP-1 mRNA and protein has been reported in different models of renal disease (34-39) and in human sclerotic glomeruli (40). The increased expression of TIMP1, a collagenase inhibitor, may be a reason for the reduced activity of collagenases and subsequent reduced breakdown of tissue collagen, leading to the observance of increased graft collagen expression and reduced collagen urine peptides in graft rejection.

Thus, altered collagen and extracellular matrix turnover in graft rejection, with altered regulation of collagenases in the graft, as seen in independent data-sets by microarray and Q-PCR, may be critical pathways that link acute rejection injury with the observed increased downstream clinical risk of chronic injury and graft fibrosis (41, 42).

### Conclusion

As is clear from the above description and experiments, non-invasive, peptidomic analysis (e.g., using mass spectrometry, followed by MRM verification) is a powerful approach to identify disease specific urine peptide biomarkers. Urine peptidomic analysis of 70 unique samples, from renal transplant patients (n=50) and controls (n=20), identified a specific panel of 53 peptides for acute rejection (AR). Peptide sequencing revealed underlying mechanisms of graft injury with a pivotal role for proteolytic degradation of uromodulin (UMOD) and a number of collagens including, COL1A2 and COL3A1. The 53 peptide panel discriminates AR in both training (n=46) and test (n=24) sets (ROC, AUC>0.9).

Integrative analysis of transcriptional signals from paired renal transplant biopsies, matched with the urine samples, reveal coordinated transcriptional changes for the corresponding genes, in addition to dysregulation of extracellular matrix proteins in AR (MMP7, SERPING1 and TIMP1). Q-PCR on an independent set of 34 transplant biopsies, with and without AR, confirms coordinated changes in expression for the corresponding genes in rejection tissue, with a 6 gene biomarker panel (COL1A2, COL3A1, UMOD, MMP7, SERPING1, TIMP1) that can also classify AR with high specificity and sensitivity (ROC, AUC 0.98).

The unique approach of integrated urine peptidomic and biopsy transcriptional analyses reveal that key collagen remodeling pathways are modulated in AR tissue, and may be the trigger for downstream chronic graft fibrosis after an AR episode. The proteolytic degradation products of the corresponding proteins in urine provide a unique non-invasive tool for diagnosis of AR.

### Methods

### Urine Samples

70 unique urine samples from 50 pediatric renal transplant recipients (20 biopsy-proven AR, 20 STA, 10 BKV), 10 age matched healthy controls (HC) and 10 pediatric patients with, non-specific proteinuria from native renal disease due to nephrotic syndrome (NS; to control for non-specific renal injury) were collected at Lucile Packard Children's Hospital at Stanford University from 2004-6. Details on patient age, gender, and other transplantation related clinical indicators are given in Table 2. Informed consent was obtained from all patients and the study was approved by the Stanford University Institutional Review Board.

### Urine Collection, Storage and Processing

Second morning void mid-stream urine samples (50-100 ml) were collected in sterile containers and were centrifuged at 2000×g for 20 minutes at room temperature within 1 hour of collection. The details of urine processing and preparation of peptide extraction and fraction is reported elsewhere (Sigdel, 2009 #9646).

### Peptidomic Data Analysis

We used the approach of ion mapping (43, 44), whereby biomarker candidate MS peaks were selected on the basis of discriminant analysis and then targeted for MS/MS sequencing analysis to obtain protein identification. We have developed an informatics platform, “MASS-Conductor” (Sigdel, 2009 #9646), which contains an integrated suite of algorithms, statistical methods, and computer applications, to allow for signal processing and statistical analysis in LCMS based urine peptide profiling. The peaks are located in the raw spectra of the MALDI data by an algorithm that looks for sites (m/z values) whose intensity is higher the estimated average background and the ˜100 surrounding sites, with peak widths ˜0.5% of the corresponding m/z value. The binned LC-MALDI MS peak data (20,937 m/z values) obtained for all 70 samples were analyzed separately for the training sample set (n=46), for discovery of discriminant biomarkers using algorithms (8) of nearest shrunken centroid (NSC), 10-fold cross validation analyses and Gaussian linear discriminant analysis (LDA). The predictive capabilities of the 53 most discriminant peptides were used to blindly test for differentiating AR, STA and BK samples in the test set (n=24). To control the number of false significant features found during NSC mining, we permutated the data set 500 times to calculate global false discovery rate (45).

### Multiple Reaction Monitoring (MRM) Assay for Peptide Marker Verification

Stable isotope labeled peptides (with a 13C-labeled amino acid) were synthesized and used as Internal Standard peptides (IS). Each urine peptide sample, prepared as described above, was diluted 10 fold with 10% acetonitrile/0.1% formic acid and spiked with the IS to final concentration 0.1 μM. Peptides were resolved in a HPLC equipped with a Polaris C18 column (50×20 mm, 3 μM, 6 min gradient elution: Buffer A: 0.1% formic acid in water Buffer B: 0.1% formic acid in acetonitrile Flow rate of 200 μl/min). A triple quadrupole mass spectrometer was used. The data was assessed and visualized by receiver-operating characteristic curve ROCR package (10).

Integrated Analysis of Peptidomic Data in Urine and Microarray Data from Matched Transplant Biopsies

Affymetirx HU133 plus 2 GeneChips on matched kidney transplant biopsies (20 AR and 20 STA) have been previously performed in the Sarwal Lab (NCBI GEO database GSE14328). Raw expression data were preprocessed and normalized using dChip software (46), and transcriptional biopsy data was analyzed for differences in expression of the corresponding UMOD and the COL genes in rejection. Additionally, we also searched for any differences in the expression of extracellular matrix proteins (TIMP1, SERPING1 and MMP7) in the rejecting graft.

### RNA Preparation and Quantitative Real-Time (RT) PCR

Total RNA was extracted from kidney biopsy samples using TRIzol reagent (Invitrogen Corporation, Carlsbad, Calif.), and later was DNasel treated and purified using the RNeasy mini kit according to the manufacturer's protocol (Qiagen, Valencia, Calif.). cDNA was synthesized from 250 ng of RNA using the RT2 First Strand Kit (SABioscience Corporation, Frederick, Md.). Quantitative real-time PCR reactions were performed on 5 ng of cDNA using RT2 SYBR Green/ROX PCR master mix and commercially available primers, PPH12000A-200 for UMOD, PPH00771A-200 for TIMP1, PPH18747E-200 for SERPING1, PPH00809E-200 for MMP7, PPH01918B-200 for COL1A2, PPH00439E-200 for COL3A1, PPH20687A-200 for COL4A1, PPH05666E-200 for 18SrRNA (SuperArray Bioscience Corporation, Frederick, Md.). All RNA samples were analyzed in duplicates and normalized relative to 18s levels.

### REFERENCES

1. Nankivell B J, Borrows R J, Fung C L, O'Connell P J, Allen R D, Chapman J R. The natural history of chronic allograft nephropathy. N Engl J Med 2003; 349:2326-33.
2. Opelz G. Influence of treatment with cyclosporine, azathioprine and steroids on chronic allograft failure. The Collaborative Transplant Study. Kidney international 1995; 52:S89-92.
3. Marsden P A. Predicting outcomes after renal transplantation—new tools and old tools. N Engl J Med 2003; 349:182-4.
4. Li B, Hartono C, Ding R, et al. Noninvasive diagnosis of renal-allograft rejection by measurement of messenger RNA for perforin and granzyme B in urine. N Engl J Med 2001; 344:947-54.
5. Sarwal M, Chua M S, Kambham N, et al. Molecular heterogeneity in acute renal allograft rejection identified by DNA microarray profiling. N Engl J Med 2003; 349:125-38.
6. Flechner S M, Kurian S M, Head S R, et al. Kidney transplant rejection and tissue injury by gene profiling of biopsies and peripheral blood lymphocytes. Am J Transplant 2004; 4:1475-89.
7. Sigdel T K, Ling X B, Lau K, et al. Urinary peptidomic analysis identifies potential biomarkers for acute rejection of renal. Clin Proteom 2009; 5:103-13.
8. Tibshirani R, Hastie T, Narasimhan B, Chu G. Diagnosis of multiple cancer types by shrunken centroids of gene expression. Proc Natl Acad Sci USA 2002; 99:6567-72.
9. Zweig M H, Campbell G. Receiver-operating characteristic (ROC) plots: a fundamental evaluation tool in clinical medicine. Clin Chem 1993; 39:561-77.
10. Sing T, Sander O, Beerenwinkel N, Lengauer T. ROCR: visualizing classifier performance in R. Bioinformatics 2005; 21:3940-1.
11. Sigdel T K, Kaushal A, Gritsenko M, et al. Shotgun Proteomics Identifies Proteins Specific for Acute Renal Transplant Rejection Under Review 2009.
12. Kottgen A, Glazer N L, Dehghan A, et al. Multiple loci associated with indices of renal function and chronic kidney disease. Nat Genet 2009.
13. Villanueva J, Shaffer D R, Philip J, et al. Differential exoprotease activities confer tumor-specific serum peptidome patterns. J Clin Invest 2006; 116:271-84.
14. Tai S S, Bunk D M, White Et, Welch M J. Development and evaluation of a reference measurement procedure for the determination of total 3,3′,5-triiodothyronine in human serum using isotope-dilution liquid chromatography-tandem mass spectrometry. Anal Chem 2004; 76:5092-6.
15. Kuyvenhoven J P, Verspaget H W, Gao Q, et al. Assessment of serum matrix metalloproteinases MMP-2 and MMP-9 after human liver transplantation: increased serum MMP-9 level in acute rejection. Transplantation 2004; 77:1646-52.
16. Adachi J, Kumar C, Zhang Y, Olsen J V, Mann M. The human urinary proteome contains more than 1500 proteins, including a large proportion of membrane proteins. Genome biology 2006; 7:R80.
17. Gonzales P A, Pisitkun T, Hoffert J D, et al. Large-Scale Proteomics and Phosphoproteomics of Urinary Exosomes. J Am Soc Nephrol 2008.
18. Coon J J, Zurbig P, Dakna M, et al. CE-MS analysis of the human urinary proteome for biomarker discovery and disease diagnostics. Proteomics Clin Appl, 2008; 2:964-73.
19. Decramer S, P ZUR, Wittke S, Mischak H, Bascands J L, Schanstra J P. Identification of urinary biomarkers by proteomics in newborns: use in obstructive nephropathy. Contrib Nephrol 2008; 160:127-41.
20. Myllyharju J, Kivirikko K I. Collagens and collagen-related diseases. Annals of medicine 2001; 33:7-21.
21. Miner J H. Renal basement membrane components. Kidney Int 1999; 56:2016-24.
22. Querejeta R, Varo N, Lopez B, et al. Serum carboxy-terminal propeptide of procollagen type I is a marker of myocardial fibrosis in hypertensive heart disease. Circulation 2000; 101:1729-35.
23. Keller F, Rehbein C, Schwarz A, et al. Increased procollagen III production in patients with kidney disease. Nephron 1988; 50:332-7.
24. Keller F, Lyreal Ser Y, Schuppan D. Raised concentrations of the carboxy terminal propeptide of type IV (basement membrane) procollagen (NC1) in serum and urine of patients with glomerulonephritis. Eur J Clin Invest 1992; 22:175-81.
25. Heickendorff L, Frost L, Madsen J K, Pedersen E B. Serum propeptides of type I and III procollagens in renal transplant recipients. A comparison of cyclosporine and azathioprine treatment. Nephron 1994; 67:203-8.
26. Berthier C C, Lods N, Joosten S A, et al. Differential regulation of metzincins in experimental chronic renal allograft rejection: potential markers and novel therapeutic targets. Kidney Int 2006; 69:358-68.
27. Melk A, Mansfield E S, Hsieh S C, et al. Transcriptional analysis of the molecular basis of human kidney aging using cDNA microarray profiling. Kidney Int 2005; 68:2667-79.
28. Edemir B, Kurian S M, Eisenacher M, et al. Activation of counter-regulatory mechanisms in a rat renal acute rejection model. BMC Genomics 2008; 9:71.
29. Zuo F, Kaminski N, Eugui E, et al. Gene expression analysis reveals matrilysin as a key regulator of pulmonary fibrosis in mice and humans. Proc Natl Acad Sci USA 2002; 99:6292-7.
30. Li Q, Park P W, Wilson C L, Parks W C. Matrilysin shedding of syndecan-1 regulates chemokine mobilization and transepithelial efflux of neutrophils in acute lung injury. Cell 2002; 111:635-46.
31. Davis J E, Moss D J. Treatment options for post-transplant lymphoproliferative disorder and other Epstein-Barr virus-associated malignancies. Tissue Antigens 2004; 63:285-92.
32. Cai S, Dole V S, Bergmeier W, et al. A direct role for C1 inhibitor in regulation of leukocyte adhesion. J Immunol 2005; 174:6462-6.
33. Woessner J F. MMPs and TIMPs: an historical perspective. Molecular biotechnology 2002; 22:1073-6085.
34. Engelmyer E, van Goor H, Edwards D R, Diamond J R. Differential mRNA expression of renal cortical tissue inhibitor of metalloproteinase-1, -2, and -3 in experimental hydronephrosis. J Am Soc Nephrol 1995; 5:1675-83.
35. Sharma A K, Mauer S M, Kim Y, Michael A F. Altered expression of matrix metalloproteinase-2, TIMP, and TIMP-2 in obstructive nephropathy. J Lab Clin Med 1995; 125:754-61.
36. Nakamura T, Takahashi T, Fukui M, et al. Enalapril attenuates increased gene expression of extracellular matrix components in diabetic rats. J Am Soc Nephrol 1995; 5:1492-7.
37. Jones C L, Buch S, Post M, McCulloch L, Liu E, Eddy A A. Pathogenesis of interstitial fibrosis in chronic purine aminonucleoside nephrosis. Kidney Int 1991; 40:1020-31.
38. Jones C L, Buch S, Post M, McCulloch L, Liu E, Eddy A A. Renal extracellular matrix accumulation in acute puromycin aminonucleoside nephrosis in rats. Am J Pathol 1992; 141:1381-96.
39. Eddy A A, Giachelli C M. Renal expression of genes that promote interstitial inflammation and fibrosis in rats with protein-overload proteinuria. Kidney Int 1995; 47:1546-57.
40. Carome M A, Striker L J, Peten E P, et al. Human glomeruli express TIMP-1 mRNA and TIMP-2 protein and mRNA. Am J Physiol 1993; 264:F923-9.
41. Weber K T. Monitoring tissue repair and fibrosis from a distance. Circulation 1997; 96:2488-92.
42. Paul L C. Chronic allograft nephropathy: An update. Kidney Int 1999; 56:783-93.
43. Fach E M, Garulacan L A, Gao J, et al. In vitro biomarker discovery for atherosclerosis by proteomics. Mol Cell Proteomics 2004; 3:1200-10.
44. Gao J, Opiteck G J, Friedrichs M S, Dongre A R, Hefta S A. Changes in the protein expression of yeast as a function of carbon source. J Proteome Res 2003; 2:643-9.
45. Ling X B, Cohen H, Jin J, Lau I, Schilling J. FDR made easy in differential feature discovery and correlation analyses. Bioinformatics 2009; 25:1461-2.
46. Li C. Automating dChip: toward reproducible sharing of microarray data analysis. BMC bioinformatics 2008; 9:231.
47. Santambrogio S, Cattaneo A, Bernascone I, et al. Urinary uromodulin carries an intact ZP domain generated by a conserved C-terminal proteolytic cleavage. Biochem Biophys Res Commun 2008; 370:410-3.
48. Strong et al., Urinary-peptide excretion by patients with and volunteers without diabetes. J Lab Clin Med. 2005 May; 145(5):239-46

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of determining a clinical transplant category of a subject who has received a kidney allograft, said method comprising:
evaluating the level of one or more peptide in a non-invasive sample from said subject to obtain a peptide signature, wherein said at least one peptide is selected from SEQ ID NO: 1 to 63; and
determining a clinical transplant category of said subject based on said peptide signature.

2. The method of claim 1, wherein the clinical transplant category is selected from: acute rejection (AR), stable allograft (STA), and BK-virus nephropathy (BK).

3. The method of claim 1, wherein the determining step comprises comparing said peptide signature to one or more reference peptide signature.

4. The method of claim 1, wherein said one or more peptide is selected from one or more of the peptides listed in SEQ ID NOs: 2, 5, 6, 7, 21, 25, 26, 31, 35, 36, 39, 42, 47, 59 and 62.

5. The method of claim 1, wherein said one or more peptide is selected from one or both of the peptides listed in: SEQ ID NO:59 and SEQ ID NO:62.

6. The method of claim 1, wherein said one or more peptide comprises the peptides listed in SEQ ID NOs: 2, 5, 6, 7, 21, 25, 26, 31, 35, 36, 39, 42, 47, 59 and 62.

7. A method of determining a clinical transplant category of a subject who has received a kidney allograft, said method comprising:
evaluating the expression level of one or more gene in a biopsy sample from said subject to obtain a gene expression signature, wherein said one or more gene comprises one or more of: COL1A2, COL3A1, MMP7, SERPING1, TIMP1 and UMOD; and
determining a clinical transplant category of said subject based on said gene expression signature.

8. The method of claim 7, wherein the clinical transplant category is selected from: acute rejection (AR), stable allograft (STA), and BK-virus nephropathy (BK).

9. The method of claim 7, wherein the determining step comprises comparing said gene expression signature to one or more reference gene expression signature.

10. The method of claim 7, wherein said one or more gene comprises COL1A2.

11. The method of claim 1, wherein said one or more gene comprises all of: COL1A2, COL3A1, MMP7, SERPING1, TIMP1 and UMOD.

12. The method of claim 7, wherein said evaluating step comprises assaying said biopsy sample for an expression product of said one or more gene, wherein said expression product is a nucleic acid transcript.

13. The method of claim 12, wherein said assaying comprises a quantitative RT-PCR assay.

14. The method of claim 7, wherein said evaluating step further comprises evaluating the level of one or more peptide in a non-invasive sample from said subject to obtain a peptide signature, wherein said at least one peptide is selected from SEQ ID NO: 1 to 63, and further wherein said clinical transplant category is based on both of said peptide signature and said gene expression signature.

15. A system for determining a clinical transplant phenotype of a subject who has received a kidney allograft, said system comprising:
a peptide level evaluation element configured for evaluating the level of one or more peptide in a non-invasive sample from said subject to obtain a peptide signature, wherein said wherein said one or more peptide is selected from SEQ ID NO: 1 to 63; and
a phenotype determination element configured for employing said peptide signature to determine a clinical transplant category of said subject.

16. The system according to claim 15, wherein said peptide level evaluation element comprises at least one reagent for assaying a non-invasive sample for the level of said one or more peptide.

17. The system according to claim 15, wherein said system further comprises:
a gene expression evaluation element configured for evaluating the expression level of one or more gene in a biopsy sample from said subject to obtain a gene expression signature, wherein said wherein said one or more gene comprises one or more of: COL1A2, COL3A1, MMP7, SERPING1, TIMP1 and UMOD;
wherein said phenotype determination element is further configured for employing said gene expression signature to determine a clinical transplant category of said subject.

18. The system of claim 17, wherein said one or more gene comprises all of: COL1A2, COL3A1, MMP7, SERPING1, TIMP1 and UMOD.

19. The system according to claim 17, wherein said phenotype determination element comprises one or more reference peptide signature and one or more reference gene expression signature to which said peptide signature and said gene expression signature are compared to determine a clinical transplant category of said subject.

20. A computer program product for determining a clinical transplant category of a subject who has received a kidney allograft, wherein said computer program product, when loaded onto a computer, is configured to employ a peptide signature from a non-invasive sample and/or a gene expression signature from a biopsy sample from said subject to determine a clinical transplant category, and provide said determined clinical transplant category to a user in a user-readable format, wherein said peptide signature comprises data for the peptide level of one or more peptides listed in SEQ ID NOs: 1 to 63, and wherein said gene expression signature comprises gene expression level data for one or more genes COL1A2, COL3A1, MMP7, SERPING1, TIMP1 and UMOD.
